(19) United States
(12) Patent Application Publication
(10) Pub. No.: US 2024/0327328 A1
(43) Pub. Date: Oct. 3, 2024

(54) PREPARATION OF 6-SUBSTITUTED MENADIONES

(71) Applicants: CENTRE NATIONAL DE LA RECHERCHE SCIENTIFIQUE, Paris (FR); UNIVERSITÉ DE STRASBOURG, STRASBOURG (FR); UNIVERSITE DE HAUTE ALSACE, MUL-HOUSE CEDEX (FR)

(72) Inventors: Elisabeth DAVIOUD-CHARVET, STRASBOURG (FR); Nathan TROMETER, LA WANTZENAU (FR); Maxime DONZEL, STRASBOURG (FR); Matthieu ROIGNANT, BISCHHEIM (FR)

(21) Appl. No.: 18/579,048

(22) PCT Filed: Jul. 13, 2022
(86) PCT No.: PCT/EP2022/069555

§ 371 Date: Jan. 12, 2024

(30) Foreign Application Priority Data
Jul. 13, 2021 (EP) 21305973.6

(51) Int. Cl. C07C 51/255

(52) U.S. Cl. CPC C07C 51/255; C07C 2602/10

(57) ABSTRACT

A process for the preparation of a compound having the following formula (I):

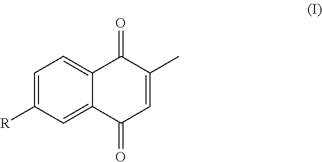

wherein R is selected from the group consisting of: halogen, (C₁-C₆)alkoxy, (C₁-C₆)alkyl, halo(C₁-C₆)alkyl, and halo(C₁-C₆)alkoxy,
said process including a step of intramolecular cyclisation, a step of bromination, a step of aromatization and a step of oxidation.

The present invention concerns a process for the preparation of 6-substituted menadiones, as well as their corresponding intermediate compounds.

The quinone structure, which is common to numerous natural products with important biological activities, is known for its ability to accept one and/or two electrons in redox processes (Elhabiri M., Sidorov P., Cesar-Rodo E., Marcou G., Lanfranchi D. A., Davioud-Charvet E., Horvath D., Varnek A. Electrochemical properties of substituted 2-methyl-1,4-naphthoquinones: redox behavior predictions. Chem.-Eur. J. 2015, 21, 3415-3424). The electron-acceptor properties of quinones, causing the formation of radical semiquinone anion or dihydroquinone dianion species responsible for in vivo oxidative stress (Sidorov P., Desta I., Chess6 M., Horvath D., Marcou G., Varnek A., Davioud-Charvet E., Elhabiri M. Redox polypharmacology is an emerging strategy to combat malarial parasites. ChemMedChem. 2016, 11, 1339-51), can be modulated by the electron-withdrawing or -donating substituents of the electroactive core. The molecular basis of quinone toxicity is the enzyme-catalyzed reduction of the quinone to semiquinone radicals, which then reduce O₂ to superoxide anion radicals and hydrogen peroxide through 1e- or 2e-transfer reactions thereby regenerating the quinone (Salmon-Chemin, L., Buisine, E., Yardley, V., Kohler, S., Debreu, M. A., Landry, V., Sergheraert, C., Croft, S. L., Krauth-Siegel, L. R., Davioud-Charvet, E. 2- and 3-substituted-1,4-naphthoquinone derivatives as subversive substrates of trypanothione reductase and lipoamide dehydrogenase from Trypanosoma cruzi: synthesis and correlation between redox cycling activities and in vitro cytotoxicity. J. Med. Chem. 2001, 44, 548-565). This redox cycling and concomitant oxygen activation leads to increased levels of reactive oxygen species (ROS) and glutathione disulfide. A well-known example is menadione (2-methyl-1,4-naphthoquinone or vitamin K3), which is a redox-cycler or a “subversive substrate” for numerous flavoproteins acting through a one-electron reduction mechanism. Menadione (2-methyl-1,4-naphthoquinone), its 6-substituted analogues (such as 6-methoxymenadione) and derivatives (like hemigossypolone, herqueidiketal, . . . ) are important examples of the broad family of 1,4-naphthoquinones (1,4-NQs), largely distributed in nature. Menadione is the parent core of vitamins K1 and K2. Vitamin K1 (phylloquinone, phytomenadione, or phytonadione) is only produced in plants; the menadione core is alkylated by a phytyl chain at the C3 position. Vitamin K2 (menaquinone) represents a series of compounds in which the phytyl side chain of phytonadione has been replaced by a side chain built up of 1 to 14 isoprenyl units.

Diversely substituted menadione derivatives display a broad pattern of various biological responses, for example, antibacterial, anticancer, antifungal, antitrypanosomal, antiinflammatory, antimalarial, or anti-Alzheimer activities, to cite a few. While there are numerous reported routes to prepare menadione derivatives alkylated at the quinone part of the 1,4-NQ core (Wang Y., Zhu S., Zou L.-H. Recent Advances in Direct Functionalization of Quinones. Eur. J. Org. Chem. 2019, 2179-2201; Donzel M., Karabiyikli D., Cotos L., Elhabiri M., Davioud-Charvet E.) Recent advances in direct C—H radical alkylation of 1,4-quinones. Eur. J. Org. Chem. 2021, Review. accepted, doi: 10.1002/ejoc.202100452), there are not many versatile methods for the regioselective preparations of synthetic naphthoquinone derivatives bearing a methyl group at C2 of the quinone moiety (east) and substituted at the phenyl ring (west part) due to the dissymmetry of the menadione molecule with its 2-methyl group (Cesar Rodo E., Feng L., Jida, M., Ehrhardt K., Bielitza M., Boilevin J., Lanzer M., Williams D. L., Lanfranchi, D. A., Davioud-Charvet, E. A platform of regioselective methodologies to access to polysubstituted 2-methyl-1,4-naphthoquinones derivatives: scope and limitations. Eur. J. Org. Chem. 2016, 11, 1982-1993).

Also, absorption spectrophotometry and/or electrochemistry (polarography, and voltammetry) are informative methods to evaluate the influence of structural effects on the reactivity. Electronic effects induced by substituents of the quinone core might cause marked shifts in the half-wave potentials, or affect the mechanism, reversibility, and/or rate of the electrode processes. If many approaches have been focused on evaluating the substitution effects on the quinone moiety (east part) of 1,4-NQs, we performed a detailed study about the influence of such substituents on the benzene ring (west part), of menadione, including at C-6.

So far, several methods were reported to describe the synthesis of polysubstituted methylnaphthols that allowed the preparation of substituted menadiones with an overall yield below 10% in a scale of several dozens of mg.

An aim of the present invention is to provide a new efficient process for the preparation of 6-substituted menadiones with a satisfying yield.

Therefore, the present invention relates to a process for the preparation of a compound having the following formula (I):

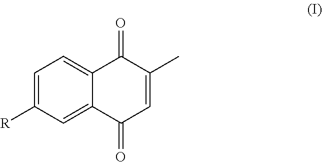

wherein R is selected from the group consisting of: halogen, (C₁-C₆)alkoxy, (C₁-C₆)alkyl, halo(C₁-C₆)alkyl, and halo(C₁-C₆)alkoxy,
said process comprising the following steps:
   a step of intramolecular cyclisation of a compound having the following formula (II):

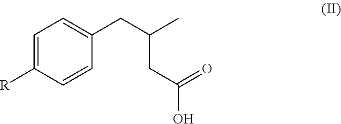

R being as defined above,
in order to obtain a compound having the following formula (III):

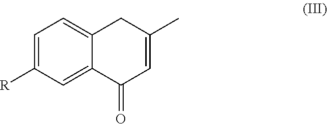

R being as defined above,
   a step of bromination of the compound having the formula (III), in order to obtain a compound having the following formula (IV):

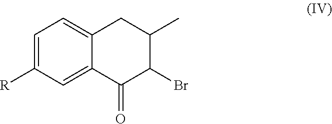

R being as defined above,
   a step of aromatization of the compound having the formula (IV), in order to obtain a compound having the following formula (V):

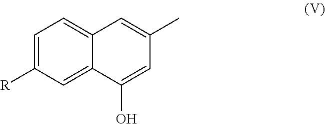

R being as defined above,
and
   a step of oxidation of the compound having the formula (V), in order to obtain the compound of formula (I).

The present invention thus concerns new synthetic methodologies in multigram scale to prepare varying menadiones substituted at C-6 in bulk, by a 4-step synthetic pathway, starting from 4-(4-substituted-phenyl)-3-methylbutanoic acids as starting materials, via tetralones as key intermediates.

The following definitions are set forth to illustrate and define the meaning and scope of the various terms used to describe the invention herein.

The expression “Cₜ-C_{z}” means a carbon-based chain which can have from t to z carbon atoms, for example C₁-C₃ means a carbon-based chain which can have from 1 to 3 carbon atoms.

The term “alkyl group” means: a linear or branched, saturated, hydrocarbon-based aliphatic group comprising, unless otherwise mentioned, from 1 to 6 carbon atoms. By way of examples, mention may be made of methyl, ethyl, n-propyl, isopropyl, butyl, isobutyl, tert-butyl or pentyl groups.

The term “halogen” means: a fluorine, a chlorine, a bromine or an iodine.

The term “alkoxy group” means: an —O-alkyl radical where the alkyl group is as previously defined. By way of examples, mention may be made of —O—(C₁-C₄)alkyl groups, and in particular the —O-methyl group, the —O-ethyl group as —O—C₃alkyl group, the —O-propyl group, the —O-isopropyl group, and as —O—C₄alkyl group, the —O-butyl, —O-isobutyl or —O-tert-butyl group.

The term “haloalkyl group” means: an alkyl group as defined above, in which one or more of the hydrogen atoms is (are) replaced with a halogen atom. By way of example, mention may be made of fluoroalkyls, in particular CF₃ or CHF₂.

The term “haloalkoxy group” means: an —O-haloalkyl group, the haloalkyl group being as defined above. By way of example, mention may be made of fluoroalkyls, in particular OCF₃ or OCHF₂.

According to an embodiment, in formula (I), R is selected from the group consisting of: F, Br, Cl, (C₁-C₆)alkoxy, (C₁-C₆)alkyl, and halo(C₁-C₆)alkyl.

According to an embodiment, in formula (I), R is selected from the group consisting of: F, Br, Cl, OMe, Me, and CF₃.

According to an embodiment, the intramolecular cyclisation step as defined above is carried out in acidic conditions, in particular in the presence of polyphosphoric acid or trifluoroacetic anhydride/triflic acid.

According to an embodiment, the intramolecular cyclisation step as defined above is carried out in a solvent such as polyphosphoric acid, methanesulfonic acid or dichloromethane, preferably in polyphosphoric acid when R═F, Br, Cl, OMe and Me and in dichloromethane when R═CF₃.

According to an embodiment, the intramolecular cyclisation step as defined above is carried out at a temperature comprised from 20° C. to 100° C., in particular from 80° C. to 100° C., preferably at 90° C. with polyphosphoric acid and at 20° C. with triflicacetic anhydride.

According to an embodiment, the intramolecular cyclisation step as defined above is carried out for a duration comprised from 1 h to 24 h, preferably for 18 hours with polyphosphoric acid and 1 hour for trifluoroacetic anhydride.

According to an embodiment, the bromination step as defined above is carried out with the addition of a brominating agent.

Examples of brominating agents include Br₂ or N-bromosuccinimide. Preferably, N-bromosuccinimide is used as brominating agent according to the present invention.

According to an embodiment, the bromination step according to the invention also includes the use of a catalyst such as para-toluenesulfonic acid.

According to an embodiment, the bromination step as defined above is carried out in a solvent such as acetic acid or dichloromethane, preferably in dichloromethane.

According to an embodiment, the bromination step as defined above is carried out at a temperature comprised from 40° C. to 55° C., preferably at 50° C.

According to an embodiment, the bromination step as defined above is carried out for a duration comprised from 1 h to 24 h, preferably for 4 hours.

According to an embodiment, the aromatization step as defined above is carried out in the presence of Li₂CO₃.

According to an embodiment, the aromatization step as defined above is carried out in a solvent such as dimethylformamide.

According to an embodiment, the aromatization step as defined above is carried out at a temperature comprised from 80° C. to 160° C., preferably at 100° C.

According to an embodiment, the aromatization step as defined above is carried out for a duration comprised from 2 h to 18 h, preferably for 4 hours.

According to an embodiment, the oxidation step as defined above is carried out in the presence of an oxidant selected from the group consisting of: phenyliodine diaceteate (PIDA), dimer [Bis(trifluoroacetoxy)iodo]benzene (PIFA), meta-chloroperoxybenzoic acid (m-CPBA), 2-iodobenzoic acid in combination with potassium hydrogenopersulphate (or peroxymonosulphate)(Oxone©) and tetrabutylammonium bromide (TBAB), Fremy's salt, and carbamide peroxide.

According to a preferred embodiment, the oxidant is the phenyliodine diaceteate (PIDA).

According to an embodiment, the oxidation step as defined above is carried out in a solvent such as acetonitrile, optionally in combination with water, nitromethane, acetone, optionally in combination with water, and hexafluoroisopropanol (HFIP).

According to a preferred embodiment, the oxidation step as defined above is carried out in acetonitrile in combination with water.

According to an embodiment, the oxidation step as defined above is carried out at a temperature comprised from −5° C. to 30° C., preferably at 25° C.

According to an embodiment, the oxidation step as defined above is carried out for a duration comprised from 30 minutes to 2 h, preferably for 1 hour.

According to an embodiment of the process according to the invention, when R is selected from the group consisting of: F, (C₁-C₆)alkoxy, (C₁-C₆)alkyl, halo(C₁-C₆)alkyl, and halo(C₁-C₆)alkoxy, the compound of formula (II) is prepared by a process comprising the following steps:
the reaction of glyoxylic acid with a compound having the following formula (VI):

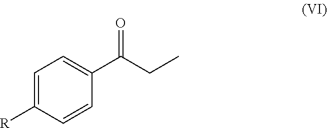

R being as defined above,
in order to obtain a compound having the following formula (VII):

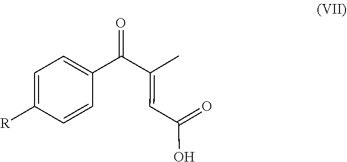

R being as defined above,
and
a step of hydrogenation of the compound having the formula (VII) in order to obtain the compound having the formula (II).

According to an embodiment, the step of reaction with glyoxylic acid as defined above is carried out in a solvent such as acetic acid or dioxane, preferably in dioxane.

According to an embodiment, the step of reaction with glyoxylic acid as defined above is carried out at a temperature comprised from 90° C. to 150° C., preferably at 100° C.

According to an embodiment, the step of reaction with glyoxylic acid as defined above is carried out for a duration comprised from 1 h to 4 h, preferably for 4 hours.

According to an embodiment, the hydrogenation step as defined above is carried out with palladium on carbon, and dihydrogen.

According to an embodiment, the hydrogenation step as defined above is carried out in a solvent such as methanol or acetic acid, preferably in acetic acid.

According to an embodiment, the hydrogenation step as defined above is carried out at a temperature comprised from 20° C. to 80° C., preferably at 70° C.

According to an embodiment, the hydrogenation step as defined above is carried out overnight.

According to an embodiment of the process according to the invention, when R is Br or Cl, the compound of formula (II) is prepared by a process comprising the following steps:
the reaction of 3-methyl-3-buten-1-ol with a compound having the following formula (VIII):

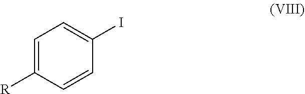

R being as defined above,
in order to obtain a compound having the following formula (IX):

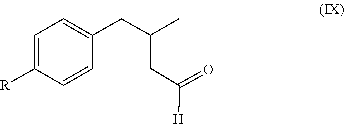

R being as defined above,
and
a step of oxidation of the compound having the formula (IX) in order to obtain the compound having the formula (II).

According to an embodiment, the step of reaction with 3-methyl-3-buten-1-ol as defined above is carried out in a solvent such as dimethylformamide.

According to an embodiment, the step of reaction with 3-methyl-3-buten-1-ol as defined above is carried out at a temperature comprised from 50° C. to 100° C., preferably at 70° C.

According to an embodiment, the step of reaction with 3-methyl-3-buten-1-ol as defined above is carried out for a duration comprised from 24 h to 72 h, preferably for 72 hours.

According to an embodiment, the oxidation of the compound having the formula (IX) as defined above is carried out with an oxidant selected from the group consisting of: periodic acid (H₅O₆), KMnO₄/H₂SO₄, and CrO₃/H₂SO₄.

According to an embodiment, the oxidation of the compound having the formula (IX) as defined above is carried out in a solvent such as acetonitrile.

According to an embodiment, the oxidation of the compound having the formula (IX) as defined above is carried out at a temperature comprised from 0° C. to 40° C., preferably from 0° C. to 25° C.

According to an embodiment, the oxidation of the compound having the formula (IX) as defined above is carried out for a duration comprised from 1 h to 4 h, preferably for 2 hours.

The present invention also relates to a compound having the following formula (I):

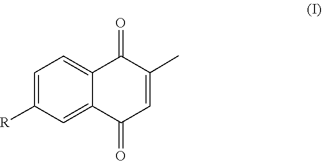

wherein R is selected from the group consisting of: Br, Cl, halo(C₁-C₆)alkyl, and halo(C₁-C₆)alkoxy,
R being preferably Br, Cl, or CF₃.

According to a preferred embodiment, the present invention relates to a compound having the following formula (I), wherein R is selected from the group consisting of: Br, halo(C₁-C₆)alkyl, and halo(C₁-C₆)alkoxy, R being preferably Br or CF₃.

The present invention also relates to a compound having the following formula (V):

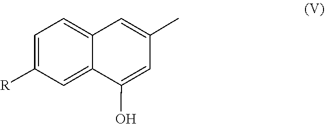

wherein R is selected from the group consisting of: F, Cl, (C₁-C₆)alkoxy, halo(C₁-C₆)alkyl, and halo(C₁-C₆)alkoxy,
R being preferably F, CF₃, Cl or OMe.

The present invention also relates to a compound having the following formula (IV):

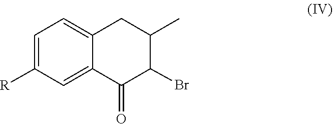

wherein R is selected from the group consisting of: F, Cl, Br, (C₁-C₆)alkoxy, (C₁-C₆)alkyl, halo(C₁-C₆)alkyl, and halo(C₁-C₆)alkoxy,
R being preferably F, Br, Cl, CF₃, Me or OMe.

The present invention also relates to a compound having the following formula (III):

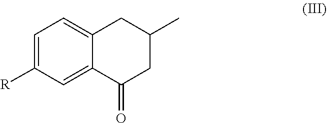

wherein R is selected from the group consisting of: F, Cl, halo(C₁-C₆)alkyl, and halo(C₁-C₆)alkoxy,
R being preferably F, Cl or CF₃.

The present invention also relates to a compound having the following formula (II):

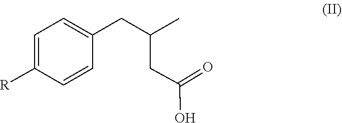

wherein R is selected from the group consisting of: Cl, (C₁-C₆)alkoxy, halo(C₁-C₆)alkyl, and halo(C₁-C₆)alkoxy,
R being preferably Cl, CF₃ or OMe.

The present invention also relates to a compound having the following formula (VII):

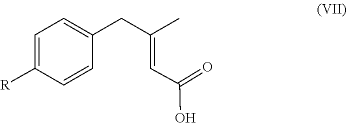

wherein R is selected from the group consisting of: Br, F, (C₁-C₆)alkoxy, halo(C₁-C₆)alkyl, and halo(C₁-C₆)alkoxy,
R being preferably F, Br, CF₃ or OMe.

According to a preferred embodiment, the present invention relates to a compound having the formula (VII), wherein R is selected from the group consisting of: halo(C₁-C₆)alkyl, and halo(C₁-C₆)alkoxy, R being preferably CF₃.

The present invention also relates to a compound being selected from the following compounds:

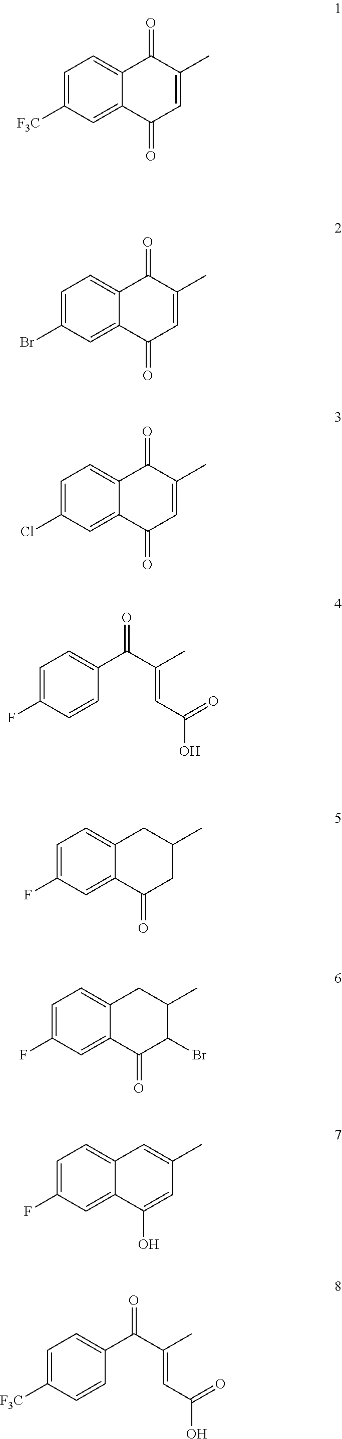

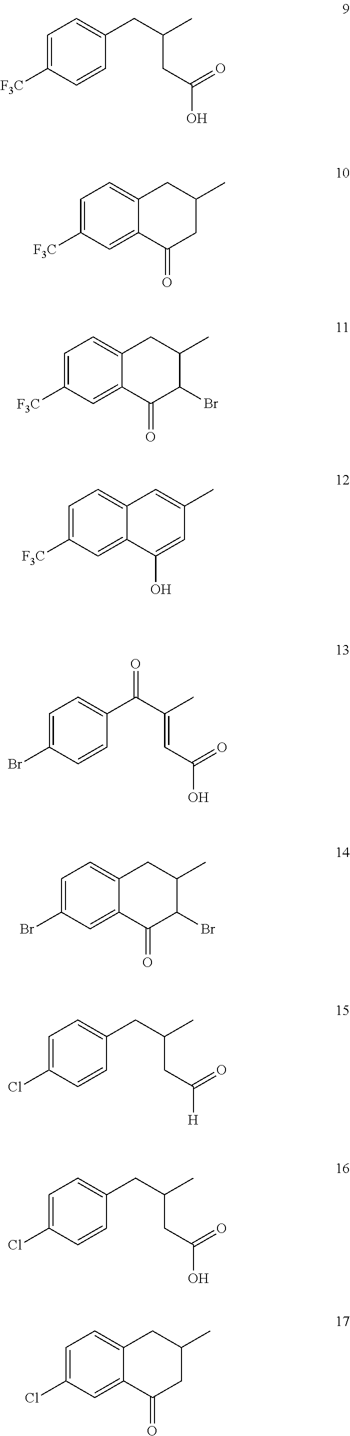

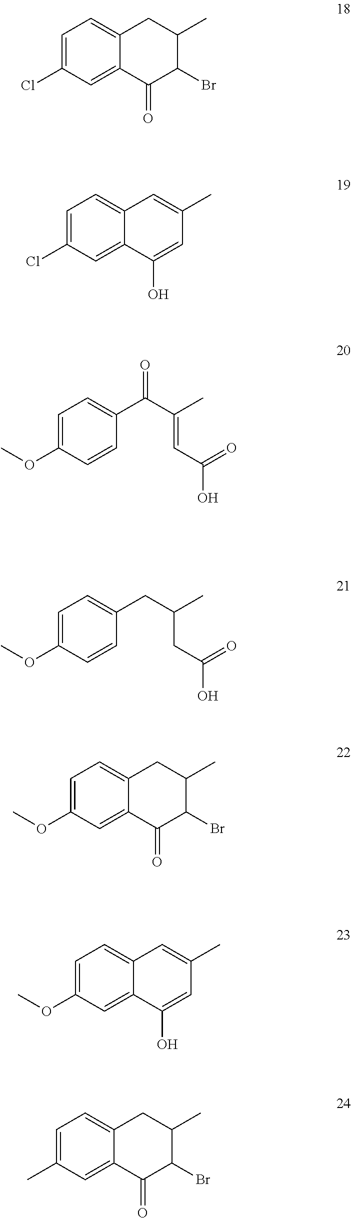

### EXAMPLES

### Example 1: Preparation of 6-fluoromenadione

The 6-fluoromenadione is prepared according to the following reaction scheme:

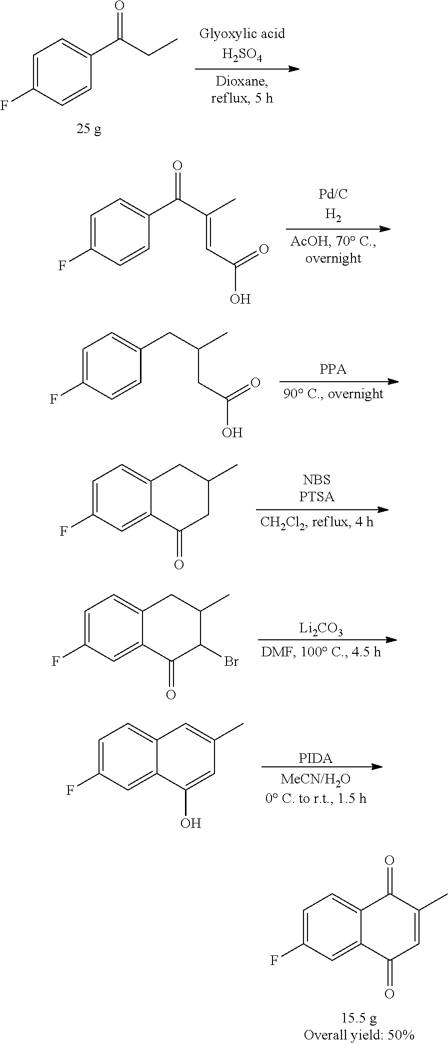

### 4-(4-fluorophenyl)-3-methyl-4-oxobut-2-enoic acid (4)

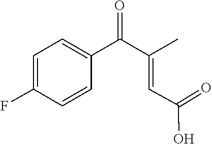

To a solution of 4′-fluoropropiophenone (0.46 mL, 3.22 mmol 1 equiv.) and glyoxylic acid monohydrate (445 mg, 4.83 mmol, 1.5 equiv.) in dioxane (4.3 mL) was added dropwise sulfuric acid (0.63 mL, 11.59 mmol, 3.6 equiv.). The solution was heated to reflux for 3 hours and followed by TLC. The resulting solution was cooled to room temperature and poured into water. The mixture was extracted with Et₂O, organic layer was repeatedly extracted with 10% K₂CO₃ aqueous solution. pH of the water layer was adjusted to acidic with a 3M HCl in water solution and extracted several times with Et₂O, the organic layer was dried over MgSO₄ and evaporated to dryness. The crude was dissolved in a minimal amount of toluene and crystallized using n-hexane giving a beige solid.

83% Yield (E:Z/85:15), beige solid. ¹H NMR (500 MHz, Acetonitrile-d3): (*refers to the isomer Z when unambiguous distinction is possible): δ 7.89-7.84 (m, 2H), 7.48* (t, J=7.1 Hz, 0.4H), 7.26-7.19 (m, 2H), 7.15* (t, J=8.7 Hz, 0.4H), 6.08 (q, J=1.6 Hz, 1H), 5.90* (s, 0.2H), 2.32 (d, J=1.6 Hz, 3H), 1.84* (s, 0.5H). ¹³C NMR (125 MHz, Acetonitrile-d3): (*refers to the isomer B when unambiguous distinction is possible) δ 197.4, 167.2, 166.7 (d, J=252.6 Hz), 152.8, 133.6 (d, J=9.5 Hz), 133.3* (d, J=3.0 Hz), 128.9*, 125.7, 118.3, 117.6*, 116.6 (d, J=22.3 Hz), 116.3* (d, J=21.8 Hz), 15.7, 12.5*. ¹⁹F NMR (471 MHz, Acetonitrile-d3): (*refers to the isomer B when unambiguous distinction is possible) δ −106.90-−107.05 (m), −114.38*-−114.47* (m). HRMS (ESI): calcd. for C₁₁H₉FNaO₃: 231.0428. Found: 231.0428 [MNa]⁺.

### 4-(4-fluorophenyl)-3-methylbutanoic acid (4-1)

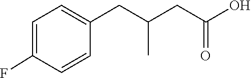

Palladium on carbon (268 mg, 0.25 mmol, 0.05 equiv.) was poured in an argon filled flask then a solution of 4-(4-fluorophenyl)-3-methyl-4-oxobut-2-enoic acid (525 mg, 2.52 mmol, 1 equiv.) in acetic acid (8.406 mL) was added to the mixture and some argon/vacuum cycles were done. Then an H₂ atmosphere was created formed and the mixture was stirred during night at 70° C. under H₂ atmosphere. The mixture was poured on celite, filtrated and washed using AcOH. The organic phase was evaporated giving a colorless oil which was purified on silica gel chromatography by flushing with DCM (1.5 column volume) and then EtOAc (2 column volume) to get rid of AcOH.

91% Yield, colorless oil. ¹H NMR (500 MHz, Chloroform-d): δ 10.94 (s, 1H), 7.12 (dd, J=8.3, 5.3 Hz, 2H), 6.97 (t, J=8.5 Hz, 2H), 2.63 (dd, J=13.6, 6.6 Hz, 2H), 2.49 (dd, J=13.6, 7.3 Hz, 2H), 2.35 (dd, J=14.7, 5.5 Hz, 2H), 2.21 (ddd, J=35.1, 14.1, 7.3 Hz, 2H), 0.97 (d, J=6.5 Hz, 3H). ¹³C NMR (125 MHz, Chloroform-d): 6179.9, 161.6 (d, J=243.7 Hz), 135.8 (d, J=3.3 Hz), 130.7 (d, J=7.7 Hz), 115.2 (d, J=21.1 Hz), 42.1, 40.9, 32.3, 19.6. ¹⁹F NMR (471 MHz, Chloroform-d) δ: −117.17-−117.25 (m). HRMS (ESI): calcd. for C₁₁H₁₂FO₂: 195.0833. Found: 195.0827 (MH).

### 7-fluoro-3-methyl-3,4-dihydronaphthalen-1(2H)-one (5)

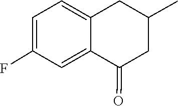

Polyphosphoric acid (10 mL) was first heated in a 60° C. water bath and then poured in a 100 mL flask containing 4-(4-fluorophenyl)-3-methylbutanoic acid (1 equiv., 441 mg, 2.25 mmol). The reaction mixture was stirred slowly at 90° C. After 5 minutes, the mixture turned yellow and after 1 h, the mixture was orange. The mixture was stirred overnight at 90° C. becoming dark orange. After TLC check, the mixture was poured on ice until PPA was completely dissolved by using sonicator and extracted with EtOAc (3 times). The reunited organic phases were washed with brine, dried over MgSO₄ and evaporated giving a yellowish-beige oil. The oil was purified on silica gel chromatography using toluene/EtOAc (9/1) giving a yellowish oil which finally crystallizes into a yellow-beige solid.

76% Yield, yellow-beige solid. ¹H NMR (500 MHz, Chloroform-d): δ 7.65 (dd, J=9.1, 2.8 Hz, 1H), 7.21 (dd, J=8.3, 5.3 Hz, 1H), 7.16 (td, J=8.3, 2.8 Hz, 1H), 2.97-2.92 (m, 1H), 2.72 (dd, J=13.0, 1.9 Hz, 1H), 2.66-2.59 (m, 1H), 2.35-2.24 (m, 2H), 1.13 (d, J=6.3 Hz, 3H). ¹³C NMR (125 MHz, Chloroform-d): δ 197.5 (d, J=1.9 Hz), 161.6 (d, J=246.1 Hz), 139.6 (d, J=3.1 Hz), 133.9 (d, J=6.2 Hz), 130.7 (d, J=7.1 Hz), 120.9 (d, J=22.2 Hz), 113.0 (d, J=21.9 Hz), 46.8, 37.4, 30.6, 21.4. ¹⁹F NMR (471 MHz, Chloroform-d): δ−115.24-−115.30 (m). HRMS (ESI): calcd. for C₁₁H₁₂FO: 179.0867. Found: 179.0877 (MH⁺). M.p.=50-54° C.

### 2-bromo-7-fluoro-3-methyl-3,4-dihydronaphthalen-1(2H)-one (6)

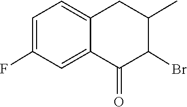

A solution of 7-fluoro-3-methyl-1,2,3,4-tetrahydronaphthalen-1-one (200 mg, 1.12 mmol, 1 equiv.) in dichloromethane (0.4 mL) was added dropwise to a solution of NBS (200 mg, 1.12 mmol, 1 equiv.) and p-toluenesulfonic acid monohydrate (22 mg, 0.11 mmol, 0.1 equiv.) in dichloromethane (1.3 mL) at 0° C. The reaction mixture was then brought to reflux for 4 h. The solid dissolves gradually during the reflux. After addition of H₂O, the organic layer was separated, and the aqueous layer was extracted with CH₂Cl₂. The combined organic layers were washed with saturated aqueous NaHCO₃ and brine, dried over anhydrous MgSO₄, and concentrated under reduced pressure.

99% Brut yield, mixture of yellow oil and crystals. The product is directly used for the next step.

### 7-fluoro-3-methylnaphthalen-1-ol (7)

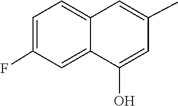

To a solution of 2-bromo-7-fluoro-3-methyl-1,2,3,4-tetrahydronaphthalen-1-one (2.3 mL, 2.3 mmol, 1 equiv.) in DMF (11.5 mL) were added lithium carbonate (187 mg, 2.53 mmol, 1.1 equiv.). The reaction mixture was stirred at 100° C. for 4.5 hours and then allowed to cool to RT. This mixture was treated with ice-cold water and extracted with diethyl ether (3 times). The combined extracts were washed with HCl (1 M), then with brine and dried (MgSO₄), and concentrated in vacuum. The crude product was purified on silica gel chromatography using cyclohexane/toluene (3/7 to 100% toluene).

71% Yield, brown-cream solid. ¹H NMR (400 MHz, Chloroform-d): δ 7.73 (dd, J=10.3, 2.6 Hz, 1H), 7.69 (dd, J=9.0, 5.5 Hz, 1H), 7.26-7.20 (m, 2H), 6.68 (s, 1H), 5.12 (s, 1H), 2.43 (s, 3H). ¹³C NMR (100 MHz, Chloroform-d): δ 160.1 (d, J=243.9 Hz), 150.8 (d, J=5.3 Hz), 135.1 (d, J=2.6 Hz), 132.0, 129.4 (d, J=8.7 Hz), 123.4 (d, J=8.8 Hz), 119.8 (d, J=1.3 Hz), 116.9 (d, J=25.3 Hz), 111.7, 105.6 (d, J=22.1 Hz), 21.7. ¹⁹F NMR (377 MHz, Chloroform-d): δ−116.06-−116.20 (m). Elemental analysis: calcd.: 74.99% C. 5.15% H. Found: 73.61% C. 5.10% H. HRMS (ESI): calcd. for C₁₁H₈FO: 175.0570. Found: 175.0565 (MH). M.p.: 119-120° C.

### 6-fluoro-2-methylnaphthalene-1,4-dione (7-1)

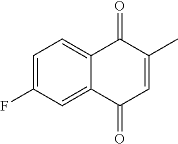

A solution of 7-fluoro-3-methylnaphthalen-1-ol (50 mg, 0.28 mmol, 1 equiv.) in acetonitrile (3.9 mL) and water (1.4 mL) was prepared. (diacetoxyiodo)benzene (192 mg, 0.6 mmol, 2.1 equiv.) was added portion wise to the stirring solution for 20 minutes at 0° C. The mixture was stirred for 30 minutes at 0° C. then at 25° C. for 1 h. A saturated NaHCO₃ solution was added to the yellow mixture and it was extracted with EtOAc. The organic phases were then washed with brine and dried over MgSO₄. The organic phase has been evaporated giving a yellow solid. The crude product was purified on silica chromatography using toluene/cyclohexane (7/3).

80% Yield, yellow solid. ¹H NMR (400 MHz, Chloroform-d): δ 8.15 (dd, J=8.6, 5.2 Hz, 1H), 7.71 (dd, J=8.5, 2.7 Hz, 1H), 7.38 (td, J=8.3, 2.7 Hz, 1H), 6.87 (q, J=1.6 Hz, 1H), 2.21 (d, J=1.6 Hz, 3H). ¹³C NMR (100 MHz, Chloroform-d): δ 184.2, 183.8 (d, J=1.6 Hz), 166.0 (d, J=257.3 Hz), 148.6, 135.7 (d, J=2.0 Hz), 134.9 (d, J=7.8 Hz), 129.8 (d, J=8.9 Hz), 128.8 (d, J=3.3 Hz), 120.8 (d, J=22.5 Hz), 112.9 (d, J=23.4 Hz), 16.5. ¹⁹F NMR (377 MHz, Chloroform-d): δ−101.07-−103.98 (m). Elemental analysis: calcd.: 69.47% C. 3.71% H. Found: 68.98% C. 3.84% H. HRMS (ESI): calcd. for C₁₁H₈FO₂:191.0509. Found: 191.0503 (MH⁺). M.p.=90-93° C.

### Example 2: Preparation of 6-methoxymenadione

The 6-methoxymenadione is prepared according to the following reaction scheme:

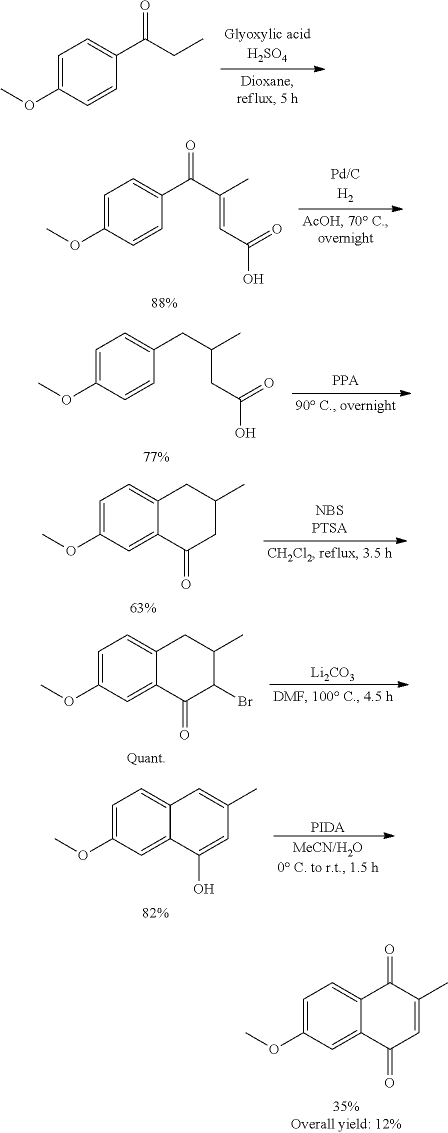

### 4-(4-methoxyphenyl)-3-methyl-4-oxobut-2-enoic acid (20)

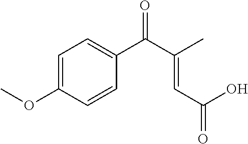 [SMILES: COc1ccc(C(=O)/C(C)=C/C(=O)O)cc1]

To a solution of 4′-methoxypropiophenone (2.35 g, 14.34 mmol, 1 equiv.) and glyoxylic acid monohydrate (1.98 g, 21.50 mmol, 1.5 equiv.) in 1,4-dioxane (18.6 mL) was added dropwise sulfuric acid (2.77 mL, 51.61 mmol, 3.6 equiv.). The solution was stirred for 5 h at reflux. The resulting solution was cooled to room temperature and poured into water. The reaction mixture was extracted with diethyl ether, then organic layer was washed 5 times with 10% K₂CO₃ aqueous solution. The pH of the aqueous layer was adjusted to acidic (pH 1) with a 3M aqueous solution of hydrochloric acid and extracted three times with diethyl ether. The organic layer was dried over MgSO₄ and evaporated under reduced pressure. Dioxane was removed by a filtration on silica gel (cyclohexane then methanol) to afford the expected product as a white solid (2.79 g, 88%) as a mixture E/Z (ratio A/B, 4/1). ¹H NMR (CDCl₃. 400 MHz): (*refers to the isomer B when unambiguous distinction is possible) δ 7.83 (d, J=8.9 Hz, 2H), 6.95 (d, J=8.9 Hz, 1.6H), 6.92* (d, J=8.9 Hz, 0.4H), 6.06 (s, 0.8H), 5.92* (s, 0.2H), 3.88 (s, 2.4H), 3.84* (s, 0.6H), 2.41 (s, 2.4H), 2.05* (0.6H). ¹³C NMR (CDCl₃, 101 MHz): (*refers to the isomer B when unambiguous distinction is possible) δ 196.5, 171.2, 164.2, 155.6*, 132.5 (2C), 128.0, 122.5, 114.2 (2C), 114.1, 55.7, 55.6*, 16.5. HRMS calculated for C₁₂H₁₂NaO₄ [M+Na]⁺: 243.062780. Found 243.062339.

### 4-(4-methoxyphenyl)-3-methylbutanoic acid (21)

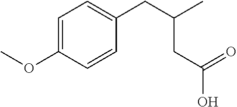

Palladium on carbon (0.65 g, 0.607 mmol, 0.05 equiv.) was poured in an Argon filled flask then a solution of 20 (2.67 g, 12.13 mmol, 1 equiv.) in acetic acid (38 mL) was added and some Ar/Vacuum cycles were done. Then a H₂ atmosphere was created by doing cycles H₂/cycles. The mixture was stirred vigorously overnight at 70° C. under H₂ atmosphere. The mixture reaction was poured on celite, filtrated and washing using acetic acid. The organic phase was evaporating to give a yellowish oil. The oil was purified by flash chromatography on silica gel (Cyclohexane, then dichloromethane, then ethyl acetate) to afford 21 as an orange oil (1.95 g, 77%). ¹H NMR (CDCl₃, 400 MHz): δ 7.08 (d, J=8.5 Hz, 2H), 6.85-6.81 (m, 2H), 3.79 (s, 3H), 2.58 (dd, J=13.6, 6.8 Hz, 1H), 2.48 (dd, J=13.6, 7.2 Hz, 1H), 2.37 (dd, J=14.6, 5.2 Hz, 1H), 2.27-2.21 (m, 1H), 2.15 (dd, J=14.6, 8.0 Hz, 1H), 0.97 (d, J=6.5 Hz, 3H). ¹³C NMR (CDCl₃, 101 MHz): 179.6, 158.1, 132.3, 130.2 (2C), 113.8 (2C), 55.4, 42.1, 40.8, 32.4, 19.7. HRMS calculated for C₁₂H₁₆NaO₃ [M+Na]⁺: 231.099165. Found 231.098129.

### 7-methoxy-3-methyl-3,4-dihydronaphthalen-1(2H)-one (21-1)

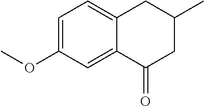

Polyphosphoric acid (40 mL) was poured in a 100 mL flask containing 21 (1.95 g, 9.36 mmol, 1 equiv.). The reaction mixture was stirred slowly overnight at 90° C. After TLC check, the mixture was poured on ice water until PPA was completely dissolved by using sonicator. Then, the aqueous phase was extracted with ethyl acetate (3 times). The reunited organic phases were washed with brine, dried over MgSO₄ and concentrated under reduced pressure. The crude product was purified by flash chromatography on silica gel (Cyclohexane/ethyl acetate, 9/1, v/v, UV) to afford LL0022 as an orange oil (1.12 g, 63%). ¹H NMR (CDCl₃, 400 MHz): δ 7.50 (d, J=2.8 Hz, 1H), 7.15 (d, J=8.4 Hz, 1H), 7.05 (dd, J=8.4, 2.8 Hz, 1H), 3.83 (s, 3H), 2.94-2.89 (m, 1H), 2.73-2.70 (m, 1H), 2.64-2.58 (m, 1H), 2.24-2.31 (m, 2H), 1.13 (d, J=6.1 Hz, 3H). ¹³C NMR (CDCl₃, 101 MHz): δ 197.9, 158.1, 136.1, 132.8, 129.8, 121.4, 108.8, 55.1, 46.8, 37.0, 30.5, 21.2. HRMS calculated for C₁₂H₁₄NaO₂ [M+Na]⁺: 213.088600. Found 213.088658.

### 2-bromo-7-methoxy-3-methyl-3,4-dihydronaphthalen-1(2H)-one (22)

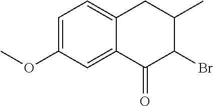

A solution of 21 (1.12 g, 5.89 mmol, 1 equiv.) in dichloromethane (2.01 mL) was added dropwise to a solution of N-bromosuccinimide (1.05 g, 5.89 mmol, 1 equiv.) and p-toluenesulfonic acid monohydrate (0.11 g, 0.59 mmol, 0.1 equiv.) in dichloromethane (6.6 mL) at 0° C. The reaction mixture was stirred at reflux for 3.5 h. After addition of water, the organic layer was separated and the aqueous layer was extracted with dichloromethane. The combined organic layers were washed with saturated aqueous NaHCO₃ and brine, dried over MgSO₄ and concentrated under reduced pressure. The crude residue was purified by flash chromatography on silica gel (Toluene, UV) to afford 22 as an orange oil and mixture of isomers (1.58 g, quant.). ¹H NMR (CDCl₃, 400 MHz): (*refers to the isomer B when unambiguous distinction is possible) δ 7.55 (d, J=2.8 Hz, 0.55H), 7.53* (d, J=2.8 Hz, 0.45H), 7.17* (s, 0.35H), 7.15 (s, 0.65H), 7.14-7.06 (m, 1H), 4.53 (dd, J=2.6, 1.2 Hz, 0.55H), 4.45-4.43* (m, 0.45H), 3.83 (s, 3H), 3.37* (dd, J=16.4, 4.2 Hz, 0.45H), 2.86 (ddd, J=16.9, 10.7, 1.0 Hz, 0.55H), 2.75-2.56 (m, 2H), 2.30-2.19 (m, 0.7H), 1.21 (d, J=6.4 Hz, 1.65H), 1.20 (d, J=6.8 Hz, 1.35H). ¹³C NMR (CDCl₃, 101 MHz): (*refers to the isomer B when unambiguous distinction is possible) δ 191.2, 190.7*, 158.7, 135.6, 133.9*, 130.7*, 130.5, 130.2*, 130.0, 123.0*, 122.9, 110.3, 110.1*, 58.8, 56.7*, 55.6, 37.6*, 35.0, 33.4*, 32.8, 19.2*, 19.1. HRMS calculated for C₁₂H₁₄BrO₂ [M+H]⁺: 269.017168. Found 269.016447.

### 7-methoxy-3-methylnaphthalen-1-ol (23)

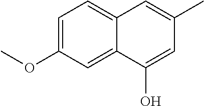

To a solution of 22 (1.58 g, 5.86 mmol, 1 equiv.) in dimethylformamide (29.3 mL) was added lithium carbonate (0.48 g, 6.45 mmol, 1.1 equiv.). The reaction mixture was stirred at 100° C. for 4.5 hours and then allowed to cool to room temperature. This reaction mixture was treated with ice-cold water and extracted with diethyl ether (3×). The combined extracts were washed a 1M solution of HCl, then with brine, dried over MgSO₄, and concentrated under reduced pressure. The crude was purified by flash chromatography on silica gel (Toluene/Cyclohexane, gradient from 9/1 to 1/0 then Toluene/ethyl acetate, 9/1, v/v, UV) to afford 23 as a white solid (49 mg, 73%). ¹H NMR (CDCl₃, 400 MHz): δ 7.63 (d, J=9.0 Hz, 1H), 7.43 (d, J=2.6 Hz, 1H), 7.17 (q, J=1.1 Hz, 1H), 7.15 (dd, J=8.9, 2.6 Hz, 1H), 5.27 (s, 1H), 3.94 (s, 3H), 2.42 (s, 4H). ¹³C NMR (CDCl₃, 101 MHz): δ 156.9, 150.4, 133.3, 130.6, 128.8, 123.5, 119.8, 119.4, 111.5, 100.0, 55.5, 21.6. HRMS calculated for C₁₂H₁₃O [M+H]⁺: 189.091006. Found 189.091766. M.p.=92-95° C.

### 6-methoxy-2-methylnaphthalene-1,4-dione (23-1)

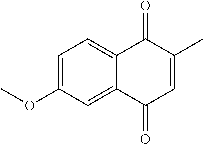

A solution of 23 (890 mg, 4.73 mmol, 1 equiv.) in a mixture of acetonitrile (65.6 mL) and water (23.0 mL) was prepared. At 0° C., (diacetoxyiodo)benzene (3.2 g, 9.93 mmol, 2.1 equiv.) was added portionwise to the stirring solution. The mixture reaction was stirred for 30 minutes at 0° C. then at room temperature for 1 h. A saturated aqueous solution of NaHCO₃ was added to the yellow mixture and it was extracted with ethyl acetate. The organic phases were washed with brine, dried over MgSO₄ and concentrated under reduced pressure. The crude mixture was purified by flash chromatography on silica gel (Toluene/ethyl acetate, gradient from 1/0 to 9/1, v/v, UV) to give a yellow solid (328 mg, 35%). ¹H NMR (CDCl₃, 400 MHz): δ 8.04 (d, J=8.6 Hz, 1H), 7.49 (d, J=2.7 Hz, 1H), 7.18 (dd, J=8.7, 2.7 Hz, 1H), 6.79 (q, J=1.6 Hz, 1H), 3.94 (s, 3H), 2.18 (d, J=1.6 Hz, 3H). ¹³C NMR (CDCl₃, 101 MHz): δ 185.2, 184.7, 164.1, 148.6, 135.4, 134.5, 129.2, 125.9, 120.4, 109.4, 56.0, 16.6. M.p.=150-153° C.

### Example 3: Preparation of 6-methylmenadione

The 6-methylmenadione is prepared according to the following reaction scheme:

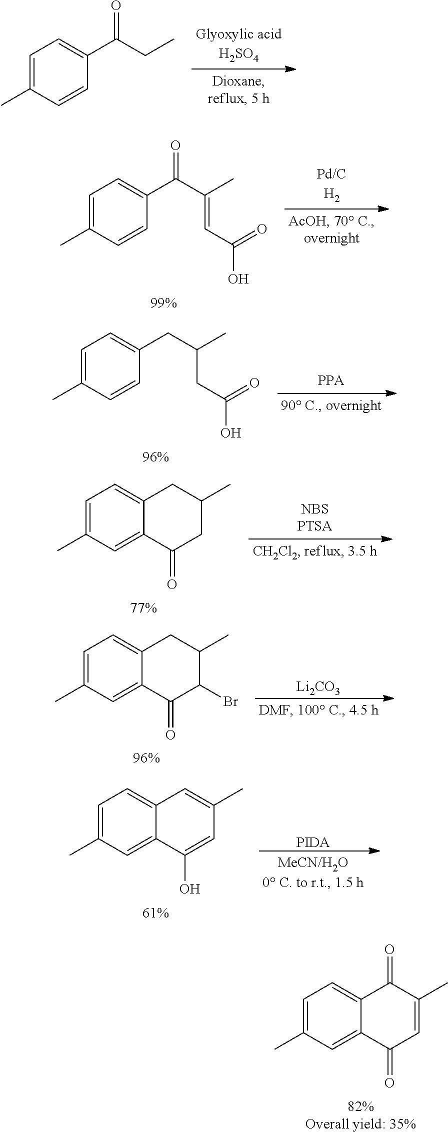

### 4-(4-methylphenyl)-3-methyl-4-oxobut-2-enoic acid (25)

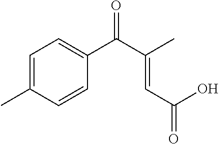

To a solution of 4′-methylpropiophenone (2 g, 13.49 mmol, 1 equiv.) and glyoxylic acid monohydrate (1.86 g, 20.24 mmol, 1.5 equiv.) in 1,4-dioxane (17.5 mL) was added dropwise sulfuric acid (2.6 mL, 48.58 mmol, 3.6 equiv.). The solution was stirred for 5 h at reflux. The resulting solution was cooled to room temperature and poured into water. The reaction mixture was extracted with diethyl ether, then organic layer was washed 5 times with 10% K₂CO₃ aqueous solution. The pH of the aqueous layer was adjusted to acidic (pH 1) with a 3M aqueous solution of hydrochloric acid and extracted three times with diethyl ether. The organic layer was dried over MgSO₄ and evaporated under reduced pressure to afford the expected product, without further purification, as a white solid (2.74 g, 99%) as a mixture E/Z. ¹H NMR (CDCl₃, 400 MHz): (*refers to the isomer B when unambiguous distinction is possible) δ 7.70 (d, J=8.1 Hz, 1.2H), 7.49* (br s, 0.8H), 7.25 (d, J=8.4 Hz, 1.2H), 7.20* (d, J=8.0 Hz, 0.8H), 6.09 (s, 0.6H), 5.87* (s, 0.4H), 2.41 (s, 1.8H), 2.36 (s, 3H), 1.98* (s, 1.2H). ¹³C NMR (CDCl₃, 101 MHz): (*refers to the isomer B when unambiguous distinction is possible) δ 197.7, 171.4, 144.6, 132.9, 130.1 (2C), 129.6, 129.5 (2C), 117.4, 21.8, 21.5*, 16.1. HRMS calculated for C₁₂H₁₂NaO₃ [M+Na]⁺: 227.067865. Found 227.068137.

### 4-(4-methylphenyl)-3-methylbutanoic acid (26)

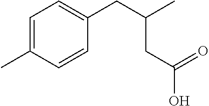

Palladium on carbon (0.69 g, 0.65 mmol, 0.05 equiv.) was poured in an Argon filled flask then a solution of 25 (2.64 g, 12.93 mmol, 1 equiv.) in acetic acid (40.5 mL) was OH added and some Ar/Vacuum cycles were done. Then a H₂ atmosphere was created by doing cycles H₂/cycles. The mixture was stirred vigorously overnight at 70° C. under H₂ atmosphere. The mixture reaction was poured on celite, filtrated and washing using acetic acid. The organic phase was evaporating to give a yellowish oil. The oil was purified by flash chromatography on silica gel (Cyclohexane, then dichloromethane, then ethyl acetate) to afford 26 as a yellowish oil (2.38 g, 96%). ¹H NMR (CDCl₃, 400 MHz): δ 7.10 (d, J=8.0 Hz, 2H), 7.06 (d, J=8.1 Hz, 2H), 2.61 (dd, J=13.5, 6.8 Hz, 1H), 2.50 (dd, J=13.5, 7.3 Hz, 1H), 2.38 (dd, J=14.8, 5.4 Hz, 1H), 2.33 (s, 3H), 2.27 (dt, J=13.3, 6.8 Hz, 1H), 2.17 (dd, J=14.8, 8.1 Hz, 1H), 0.99 (d, J=6.5 Hz, 3H). ¹³C NMR (CDCl₃, 101 MHz): δ 179.7, 137.1, 135.7, 129.2 (2C), 129.1 (2C), 42.6, 40.9, 32.3, 21.1, 19.7. HRMS calculated for C₁₂H₁₆NaO₂ [M+Na]⁺: 215.104250. Found 215.104381.

### 7-methyl-3-methyl-3,4-dihydronaphthalen-1(2H)-one (27)

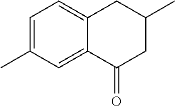

Polyphosphoric acid (30 mL) was poured in a 100 mL flask containing 26 (2.3 g, 11.70 mmol, 1 equiv.). The reaction mixture was stirred slowly overnight at 90° C. After TLC check, the mixture was poured on ice water until PPA was completely dissolved by using sonicator. Then, the aqueous phase was extracted with ethyl acetate (3 times). The reunited organic phases were washed with brine, dried over MgSO₄ and concentrated under reduced pressure. The crude product was purified by flash chromatography on silica gel (Toluene/ethyl acetate, 8/2, v/v) to afford 27 as a white solid (1.58 g, 77%). ¹H NMR (CDCl₃, 400 MHz): δ 7.81 (s, 1H), 7.27 (dd, J=7.5, 2.0 Hz, 1H), 7.11 (d, J=7.8 Hz, 1H), 2.97-2.86 (m, 1H), 2.69 (dd, J=12.8, 1.9 Hz, 1H), 2.61 (dd, J=16.4, 10.5 Hz, 1H), 2.34 (s, 3H), 2.31-2.21 (m, 2H), 1.11 (d, J=6.2 Hz, 3H). ¹³C NMR (CDCl₃, 101 MHz): δ 198.8, 141.0, 136.3, 134.5, 132.0, 128.8, 127.1, 47.3, 37.7, 30.7, 21.4, 21.0. HRMS calculated for C₁₂H₁₄NaO [M+Na]⁺: 197.093686. Found 197.094299. M.p.=47-48° C.

### 2-bromo-7-methyl-3-methyl-3,4-dihydronaphthalen-1(2H)-one (24)

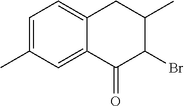

A solution of 27 (150 mg, 0.86 mmol, 1 equiv.) in dichloromethane (0.31 mL) was added dropwise to a solution of N-bromosuccinimide (153.2 mg, 0.86 mmol, 1 equiv.) and p-toluenesulfonic acid monohydrate (16.6 mg, 0.086 mmol, 0.1 equiv.) in dichloromethane (0.96 mL) at 0° C. The reaction mixture was stirred at reflux for 3.5 h. After addition of water, the organic layer was separated and the aqueous layer was extracted with dichloromethane. The combined organic layers were washed with saturated aqueous NaHCO₃ and brine, dried over MgSO₄ and concentrated under reduced pressure. The crude residue was purified by flash chromatography on silica gel (Toluene/cyclohexane, 4/6, UV) to afford 24 as an orange oil and mixture of isomers (210 mg, 96%). ¹H NMR (CDCl₃, 400 MHz): (*refers to the isomer B when unambiguous distinction is possible) δ 7.90-7.87 (m, 1H), 7.36-7.32 (m, 1H), 7.15 (m, 1H), 4.53 (dd, J=2.7, 1.4 Hz, 0.7H), 4.44* (m, 0.3H), 3.40* (dd, 0.3H, J=16.8, 4.5 Hz), 2.90 (m, 0.70H), 2.72 (m, 1H), 2.62* (m, 0.3H), 2.37 (s, 3H), 2.25 (m, 0.7H), 1.22-1.20 (m, 3H). ¹³C NMR (CDCl₃, 101 MHz): (*refers to the isomer B when unambiguous distinction is possible) δ 191.3, 190.8*, 140.0, 138.4*, 136.8, 135.5*, 135.2, 129.6*, 129.1*, 129.1, 128.7, 128.7, 128.3*, 128.2*, 59.1, 57.0*, 37.4*, 34.7, 33.7*, 33.1, 21.8*, 21.0, 19.2*, 19.1. HRMS calculated for C₁₂H₁₃BrNaO [M+Na]⁺: 275.004198. Found 275.004086.

### 7-methyl-3-methylnaphthalen-1-ol (28)

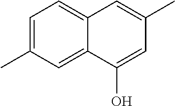

To a solution of 24 (2.32 g, 9.18 mmol, 1 eq) in dimethylformamide (54.7 mL) was added lithium carbonate (0.75 g, 10.09 mmol, 1.1 equiv.). The reaction mixture was stirred at 100° C. for 4.5 h and then allowed to cool to room temperature. This reaction mixture was treated with ice-cold water and extracted with diethyl ether (3×). The combined extracts were washed a 1M solution of HCl, then with brine, dried over MgSO₄, and concentrated under reduced pressure. The crude was purified by flash chromatography on silica gel (Toluene, UV) to afford 28 as a brown solid (971 mg, 61%). ¹H NMR (CDCl₃, 400 MHz): δ 7.86 (s, 1H), 7.62 (d, J=8.3 Hz, 1H), 7.29 (dd, J=8.4, 1.8 Hz, 1H), 7.18 (s, 1H), 6.64 (d, J=1.7 Hz, 1H), 5.04 (m, 1H), 2.51 (s, 3H), 2.43 (s, 3H). ¹³C NMR (CDCl₃, 101 MHz): δ 150.7, 134.9, 134.2, 133.3, 128.9, 127.1, 122.8, 120.3, 119.8, 111.0, 21.9, 21.8. HRMS calculated for C₁₂H₁₃O [M+H]⁺: 173.096091. Found 173.096314. M.p.=93-96° C.

### 6-methyl-2-methylnaphthalene-1,4-dione (29)

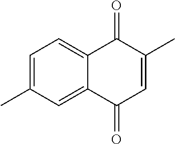

A solution of 28 (970 mg, 5.64 mmol, 1 equiv.) in a mixture of acetonitrile (115.2 mL) and water (35.2 mL) was prepared. At 0° C. (diacetoxyiodo)benzene (2.18 g, 6.77 mmol, 2.1 equiv.) was added portionwise to the stirring solution. The mixture reaction was stirred for 30 minutes at 0° C. then at room temperature for 1 h. A saturated aqueous solution of NaHCO₃ was added to the yellow mixture and it was extracted with ethyl acetate. The organic phases were washed with brine, dried over MgSO₄ and concentrated under reduced pressure. The crude mixture was purified by flash chromatography on silica gel (Toluene/Cyclohexane, 8/2, v/v, UV) to give a yellow solid (857 mg, 82%). ¹H NMR (CDCl₃, 400 MHz): δ 7.98 (d, J=7.9 Hz, 1H), 7.85 (dd, J=1.2, 0.6 Hz, 1H), 7.50 (ddd, J=8.0, 1.8, 0.9 Hz, 1H), 6.79 (q, J=1.6 Hz, 1H), 2.48 (s, 3H), 2.18 (d, J=1.6 Hz, 3H). ¹³C NMR (CDCl₃, 101 MHz): δ 185.5, 185.4, 148.3, 144.9, 135.6, 134.4, 132.3, 130.1, 126.8, 126.5, 21.9, 16.6. HRMS calculated for C₁₂H₁₁O₂ [M+H]⁺: 187.075356. Found 187.076313. M.p.=130-132° C.

### Example 4: Preparation of 6-trifluoromenadione

The 6-trifluoromenadione is prepared according to the following reaction scheme:

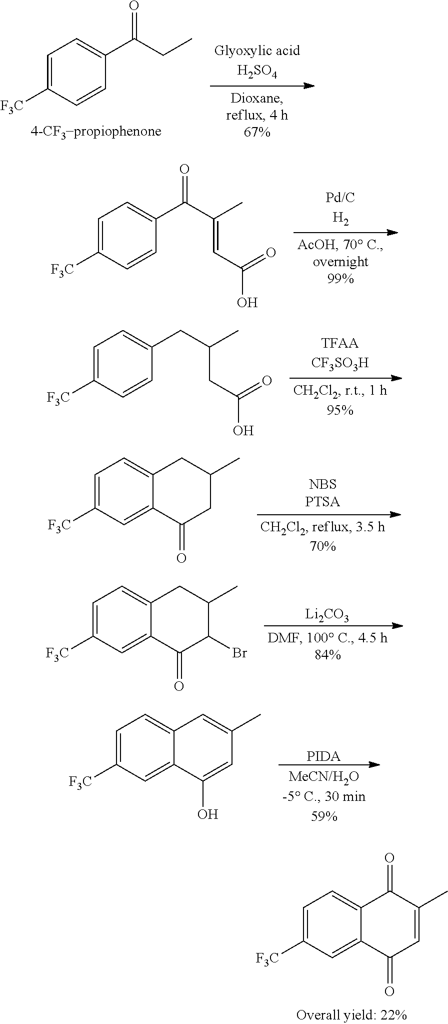

### 4-(4-trifluorophenyl)-3-methyl-4-oxobut-2-enoic acid (8)

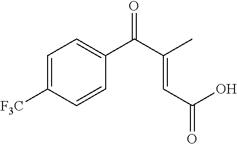 [SMILES: C/C(=C\C(=O)O)C(=O)c1ccc(C(F)(F)F)cc1]

To a solution of 4′-trifluoromethylpropiophenone (5 g, 24.73 mmol, 1 equiv.) and glyoxylic acid monohydrate (2.75 g, 37.09 mmol, 1.5 equiv.) in 1,4-dioxane (31.6 mL) was added dropwise sulfuric acid (4.77 mL, 89.03 mmol, 3.6 equiv.). The solution was stirred for 4 h at reflux. The resulting solution was cooled to room temperature and poured into water. The reaction mixture was extracted with diethyl ether, then organic layer was washed 5 times with 10% K₂CO₃ aqueous solution. The pH of the aqueous layer was adjusted to acidic (pH 1) with a 3M aqueous solution of hydrochloric acid and extracted three times with diethyl ether. The organic layer was dried over MgSO₄ and evaporated under reduced pressure to afford the expected product, without further purification, as a white solid (4.29 g, 67%) as a mixture E/Z (ratio A/B, 4/1, *refers to the isomer B when unambiguous distinction is possible). ¹H NMR (CDCl₃, 400 MHz): δ 11.34 (br s, 1H), 7.89 (d, AB syst, J=8.0 Hz, 2H), 7.74 (d, AB syst, J=8.1 Hz, 2H), 7.66-7.58* (m, 1H), 6.17 (d, J=1.6 Hz, 1H), 5.88* (s, 0.25H), 2.43 (s, 3H), 1.92* (s, 0.75H). ¹⁹F NMR (377 MHz, CDCl₃): δ −62.86*, −63.24. ¹³C NMR (101 MHz, CDCl₃): δ 196.8, 171.9*, 170.0, 168.8*, 153.7, 140.2*, 138.7, 134.8 (q, ²J_{C-F}=32.8 Hz), 131.8* (q, ²J_{C-F}=32.9 Hz), 130.1 (2C), 127.6*, 126.4*, 125.8 (q, ³J_{C-F}=3.7 Hz, 2C), 125.6*, 123.9 (d, ¹J_{C-F}=272.7 Hz), 123.5 (d, ¹J_{C-F}=272.8 Hz), 119.5*, 117.0, 106.9*, 15.7, 12.6*. HRMS (ESI+): calculated for C₁₂H₉F₃NaO₃ [M+Na]⁺: 281.039600. Found 281.039775.

### 4-(4-trifluorophenyl)-3-methylbutanoic acid (9)

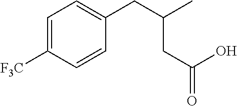 [SMILES: CC(CC(=O)O)Cc1ccc(C(F)(F)F)cc1]

Palladium on carbon (0.83 g, 0.78 mmol, 0.05 equiv.) was poured in an Argon filled flask then a solution of 8 (4.04 g, 15.65 mmol, 1 equiv.) in acetic acid (60.9 mL) was added and some Ar/Vacuum cycles were done. Then a H₂ atmosphere was created by doing cycles H₂/cycles. The mixture was stirred vigorously overnight at 70° C. under H₂ atmosphere. The mixture reaction was poured on celite, filtrated and washing using acetic acid. The organic phase was evaporating to give 9, without further purification, as a yellowish oil (3.81 g, 99%). ¹H NMR (400 MHz, CDCl₃): δ 11.97 (s, 1H), 7.56 (d, AB syst, J=7.9 Hz, 2H), 7.30 (d, AB syst, J=7.9 Hz, 2H), 2.75 (dd, J=13.5, 6.0 Hz, 1H), 2.59 (dd, J=13.5, 7.1 Hz, 1H), 2.43-2.29 (m, 2H), 2.24 (dd, J=13.9, 6.4 Hz, 1H), 1.01 (d, J=6.2 Hz, 3H). ¹⁹F NMR (377 MHz, CDCl₃): δ −62.34. ¹³C NMR (101 MHz, CDCl₃): δ 179.9, 144.4, 129.6 (2C), 128.7 (q, ²J_{C-F}=33.3 Hz), 125.4 (d, ³J_{C-F}=3.7 Hz, 2C), 124.5 (q, ¹J_{C-F}=271.8 Hz), 42.7, 40.9, 32.0, 19.6. HRMS (ESI+): calculated for C₁₂H₁₄F₃O₂ [M+H]⁺: 247.094041. Found 247.093769.

### 7-trifluoro-3-methyl-3,4-dihydronaphthalen-1(2H)-one (10)

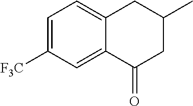 [SMILES: CC1CC(=O)c2cc(C(F)(F)F)ccc2C1]

In a round bottom flask, the acid 9 (3.43 g, 13.93 mmol, 1 equiv.) was dissolved in dichloromethane (34 mL) then TFAA (11.62 mL, 83.58 mmol, 6 equiv.) was added followed by triflic acid (1.86 mL, 20.89 mmol, 1.5 equiv.). The mixture reaction was stirred for 1 h. After this time, the mixture was treated with water and extracted with dichloromethane. The organic phase was washed with a saturated aqueous solution of NaHCO₃, dried over MgSO₄ and concentrated under reduced pressure. The crude mixture was purified by flash chromatography on silica gel (Toluene/Ethyl acetate, 8/2, v/v, UV) to afford the expected product 10 as a white solid (3.04 g, 95%). ¹H NMR (CDCl₃, 400 MHz): δ 8.27 (s, 1H), 7.68 (dd, J=8.0, 2.0 Hz, 1H), 7.37 (d, J=8.0 Hz, 1H), 3.03 (br d, 1H, J=17.5 Hz), 2.78-2.69 (m, 2H), 2.39-2.32 (m, 2H), 1.16 (2d, 3H, J=6.3 Hz). ¹⁹F NMR (377 MHz, CDCl₃): δ −62.7. ¹³C NMR (101 MHz, CDCl₃): δ 197.2, 147.4, 132.6, 129.8, 129.7 (q, ³J_{C-F}=3.6 Hz), 129.3, 124.3 (q, ³J_{C-F}=3.6 Hz), 124.0 (d, ¹J_{C-F}=272.2 Hz), 46.9, 37.9, 30.3, 21.3. HRMS (ESI+): calculated for C₁₂H₁₂F₃O [M+H]⁺: 229.083476. Found 229.073453. M.p.: 55-58° C.

### 2-bromo-7-trifluoro-3-methyl-3,4-dihydronaphthalen-1(2H)-one (11)

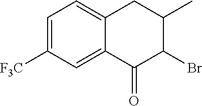 [SMILES: CC1Cc2ccc(C(F)(F)F)cc2C(=O)C1Br]

A solution of 10 (609 mg, 2.67 mmol, 1 equiv.) in dichloromethane (1.2 mL) was added dropwise to a solution of N-bromosuccinimide (570.0 mg, 3.2 mmol, 1 equiv.) and p-toluenesulfonic acid monohydrate (51.5 mg, 0.27 mmol, 0.1 equiv.) in dichloromethane (3.1 mL) at 0° C. The reaction mixture was stirred at reflux for 3.5 h. After addition of water, the organic layer was separated and the aqueous layer was extracted with dichloromethane. The combined organic layers were washed with saturated aqueous NaHCO₃ and brine, dried over MgSO₄ and concentrated under reduced pressure. The crude residue was purified by flash chromatography on silica gel (Toluene/cyclohexane, 7/3, v/v, UV) to afford 11 as a yellowish oil (576 mg, 70%) and mixture of isomers (mixture A/B (ratio 1.8/1, *refers to the isomer B when unambiguous distinction is possible). ¹H NMR (CDCl₃, 400 MHz): δ 8.34 (2s, 1H), 7.74 (m, 1H), 7.41 (d, J=7.8 Hz, 0.35H), 7.40 (d, J=8.1 Hz, 0.65H), 4.56 (br s, 0.65H), 4.46* (d, J=5.9 Hz, 0.35H), 3.52* (dd, J=17.4, 4.6 Hz, 0.35H), 2.99 (dd, J=17.5, 10.8 Hz, 0.65H), 2.83 (2dd, J=16.9, 11.5 Hz, 1H), 2.70-2.64* (m, 0.35H), 2.33-2.27 (m, 0.65H), 1.24 (d, J=6.4 Hz, 1.95H), 1.20* (d, J=7.0 Hz, 1.05H). ¹⁹F NMR (377 MHz, CDCl₃): δ −62.83. ¹³C NMR (CDCl₃, 101 MHz): δ 189.9, 189.5*, 146.4, 144.8*, 130.5* (q, ³J_{C-F}=3.7 Hz), 130.3*, 130.2 (q, ³J_{C-F}=3.6 Hz), 130.0, 129.8, 129.7, 125.9 (d, ³J_{C-F}=4.2 Hz), 125.5* (d, ³J_{C-F}=3.6 Hz), 123.7 (q, ¹J_{C-F}=272.3 Hz), 58.0, 55.2*, 36.8*, 34.3, 33.6*, 33.4, 18.9, 18.8*. HRMS (ESI+): calculated for C₁₂H₁₁BrF₃O [M+H]⁺: 306.993988. Found 306.993449.

### 7-trifluoro-3-methylnaphthalen-1-ol (12)

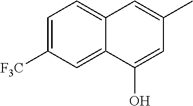 [SMILES: Cc1cc(O)c2cc(C(F)(F)F)ccc2c1]

To a solution of 11 (477.8 mg, 1.56 mmol, 1 equiv.) in dimethylformamide (9.3 mL) was added lithium carbonate (126.5 g, 1.71 mmol, 1.1 equiv.). The reaction mixture was stirred at 100° C. for 4.5 h and then allowed to cool to room temperature. This reaction mixture was treated with ice-cold water and extracted with diethyl ether (3×). The combined extracts were washed a 1M solution of HCl, then with brine, dried over MgSO₄, and concentrated under reduced pressure. The crude was purified by flash chromatography on silica gel (Toluene/cyclohexane, 7/3, v/v, UV) to afford 12 as a brown solid (319 mg, 91%). ¹H NMR (CDCl₃, 400 MHz): δ 8.48 (s, 1H), 7.79 (d, AB syst, J=8.6 Hz, 1H), 7.60 (dd, AB syst, J=8.7, 1.9 Hz, 1H), 7.25 (s, 1H), 6.71 (s, 1H), 5.43 (s, 1H), 2.46 (s, 3H). ¹⁹F NMR (377 MHz, CDCl₃): δ −62.02. ¹³C NMR (CDCl₃, 101 MHz): δ 151.9, 138.8, 136.1, 128.0, 126.3 (d, ²J_{C-F}=33.3 Hz), 124.8 (d, ¹J_{C-F}=273.7 Hz), 122.2 (q, ³J_{C-F}=3.2 Hz), 121.7, 120.1 (q, ³J_{C-F}=4.5 Hz), 119.8, 112.0, 22.0. HRMS (ESI+): calculated for C₁₂H₁₀F₃O [M+H]⁺: 227.067826. Found 227.067355. M.p.: 88-91° C.

### 7-trifluoro-3-methylnaphthalen-1-ol (1)

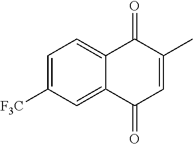 [SMILES: CC1=CC(=O)c2cc(C(F)(F)F)ccc2C1=O]

A solution of 12 (2.45 g, 10.83 mmol, 1 equiv.) in a mixture of acetonitrile (192.9 mL) and water (67.7 mL) was prepared. At 0° C., (diacetoxyiodo)benzene (4.19 g, 13.0 mmol, 2.1 equiv.) was added portionwise to the stirring solution. The mixture reaction was stirred for 30 minutes at −5° C. A saturated aqueous solution of NaHCO₃ was added to the yellow mixture and it was extracted with ethyl acetate. The organic phases were washed with brine, dried over MgSO₄ and concentrated under reduced pressure. The crude mixture was purified by flash chromatography on silica gel (Toluene/Cyclohexane, 6/4, v/v, UV) to give a yellow solid (1.54 g, 59%). ¹H NMR (CDCl₃, 400 MHz): δ 8.33, 8.24 (d, AB syst, J=8.1 Hz, 2H), 7.98 (dd, AB syst, J=8.1, 1.8 Hz, 1H), 6.93 (q, J=1.6 Hz, 1H), 2.23 (d, J=1.5 Hz, 3H). ¹⁹F NMR (377 MHz, CDCl₃): δ −63.38. ¹³C NMR (CDCl₃, 101 MHz): δ 184.5, 183.6, 148.8, 136.0, 135.4 (d, ²J_{C-F}=33.3 Hz), 134.3, 132.7, 130.0 (q, ³J_{C-F}=3.7 Hz), 127.4, 123.5 (q, ³J_{C-F}=3.9 Hz), 123.3 (d, ¹J_{C-F}=273.3 Hz), 16.6. HRMS (ESI+): calculated for C₁₂H₈F₃O₂[M+H]⁺: 241.047091. Found 241.046381. M.p.: 97-99° C.

### Example 5: Preparation of 6-bromomenadione

The 6-bromomenadione is prepared according to the following reaction scheme:

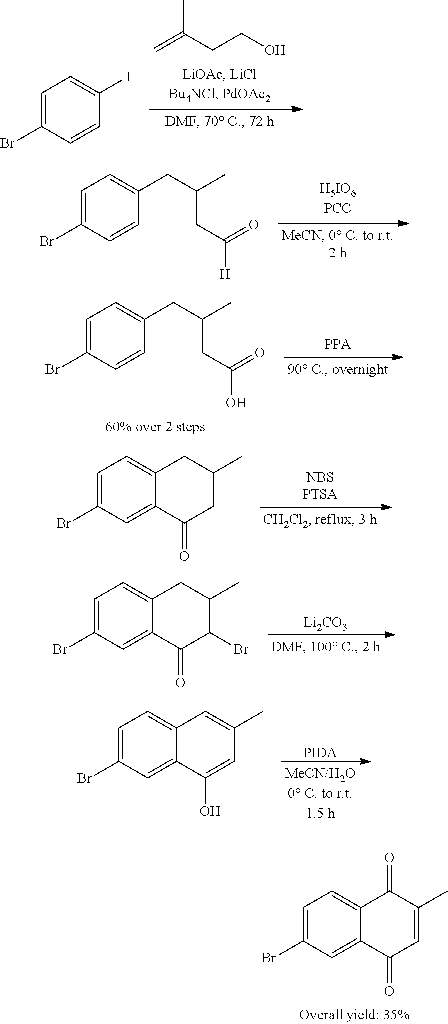

### 4-(4-bromophenyl)-3-methyl-4-oxobut-2-enoic acid (13)

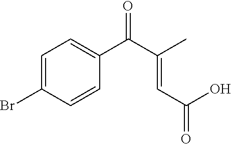

To a solution of 4′-bromopropiophenone (2 g, 9.39 mmol, 1 equiv.) and glyoxylic acid monohydrate (1.3 g, 14.08 mmol, 1.5 equiv.) in dioxane (12.2 mL) was added dropwise sulfuric acid (1.81 mL, 33.79 mmol, 3.6 equiv.). The solution was stirred overnight at reflux. The resulting solution was cooled to room temperature and poured into water. The reaction mixture was extracted with diethyl ether, then organic layer was washed 5 times with 10% K₂CO₃ aqueous solution. The pH of the aqueous layer was adjusted to acidic (pH 1) with a 3M aqueous solution of hydrochloric acid and extracted three times with diethyl ether. The organic layer was dried over MgSO₄ and evaporated under reduced pressure. Dioxane was removed by a filtration on silica gel (cyclohexane then methanol) to afford the expected product as a white solid (2.27 g, 90%) as a mixture E/Z (ratio A/B, 4/1). ¹H NMR (CDCl₃, 400 MHz): (*refers to the isomer B when unambiguous distinction is possible) δ 7.67 (d, AB syst, J=8.6 Hz, 2H), 7.62 (d, AB syst, J=8.6 Hz, 2H), 7.52* (d, AB syst, J=8.6 Hz, 0.4H), 7.34* (d, AB syst, J=8.5 Hz, 0.4H), 6.15 (br d, J=1.4 Hz, 1H), 5.86* (br d, J=1.0 Hz, 0.2H), 2.42 (d, J=1.3 Hz, 3H), 1.91* (d, J=0.8 Hz, 0.6H). ¹³C NMR (CDCl₃, 101 MHz): δ (*refers to the isomer B when unambiguous distinction is possible) 196.8, 171.0, 168.6*, 154.3, 135.5*, 134.2, 132.2 (2C), 132.0*, 131.4 (2C), 130.3*, 129.0*, 127.6, 124.3*, 116.8, 106.8*, 16.1, 12.6*. HRMS calculated for C₁₁H₉BrNaO₃ [M+Na]⁺: 290.962727. Found 290.963532.

### 4-(4-bromophenyl)-3-methylbutanoic acid (30)

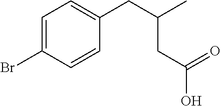

30 was synthetized according to Sleebs procedure (Sleebs, B. E.; Kersten, W. J. A.; Kulasegaram, S.; Nikolakopoulos, G.; Hatzis, E.; Moss R. M.; Parisot, J. P.; Yang, H.; Czabotar, P. E.; Fairlie, W. D.; Lee, E. F.; Adams, J. M.; Chen, L; Van Delft, M. F.; Lowes, K. N., Wei, A.; Huang, D. C. S.; Colman, P. M.; Street, I. P.; Baell, J. B.; Watson, K.; Lessene, G. J. Med. Chem. 2013, 59, 5514-5540). A mixture of 1-bromo-4-iodobenzene (10.0 g, 35.35 mmol, 1 equiv.), lithium acetate (5.8 g, 87.97 mmol, 2.49 equiv.), lithium chloride (1.5 g, 35.39 mmol, 1 equiv.), 3-methyl-3-buten-1-ol (3.6 mL, 35.35 mmol, 1 equiv.), tetrabutylammonium chloride hydrate (21.0 g, 70.77 mmol, 2 equiv.) and palladium diacetate (0.45 g, 2 mmol, 0.057 equiv.) in dimethylformamide (68 mL) was stirred at 70° C. for 72 h. After this time, the reaction mixture was quenched with a saturated aqueous solution of ammonium chloride. Then, the reaction mixture was extracted three times with diethyl ether. The combined organic layers were washed with brine, dried over MgSO₄ and concentrated under reduced pressure. No further purification was required and the product was used in the next step directly. 280 mL of acetonitrile was added to periodic acid (8.1 g, 35.46 mmol, 1.09 equiv.) and the resulting mixture was stirred for 15 min. At 0° C., a solution of 4-(4-bromophenyl)-3-methylbutanal (7.9 g, 32.68 mmol, 1 equiv.) in acetonitrile (35 mL) was added followed by pyridinium chlorochromate (0.14 g, 0.65 mmol, 0.02 equiv.) in 35 mL of acetonitrile. The reaction mixture was then stirred at room temperature for 3.5 h. After this time, ethyl acetate was added. The organic phase was washed with a 1:1 water/brine mixture, a saturated aqueous of sodium hydrogen sulfate solution and brine. It was then dried over MgSO₄ and concentrated under reduced pressure. The crude product was purified by flash chromatography on silica gel (Dichloromethane/methanol, gradient from 98/2 to 95/5, v/v, UV) to afford 30 as an oil. ¹H NMR (CDCl₃, 400 MHz): δ 7.44-7.37 (m, 2H), 7.04 (d, J=8.3 Hz, 2H), 2.62 (dd, J=13.5, 6.4 Hz, 1H), 2.47 (dd, J=13.5, 7.3 Hz, 1H), 2.35 (dd, J=14.3, 5.2 Hz, 1H), 2.30-2.15 (m, 3H), 0.97 (d, J=6.4 Hz, 3H). ¹³C NMR (CDCl₃, 101 MHz): δ 179.2, 139.1, 131.5 (2C), 131.1 (2C), 120.1, 42.3, 40.7, 32.1, 19.6. HRMS calculated for C₁₁H₁₂BrO₂ [M−H]⁻: 255.002615. Found 255.001185.

### 7-bromo-3-methyl-3,4-dihydronaphthalen-1(2H)-one (31)

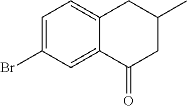

Polyphosphoric acid (80 mL) was poured in a 250 mL flask containing 30 (5.46 g, 21.23 mmol, 1 equiv.). The reaction mixture was stirred slowly overnight at 90° C. After TLC check, the mixture was poured on ice water until PPA was completely dissolved by using sonicator. Then, the aqueous phase was extracted with ethyl acetate (3 times). The reunited organic phases were washed with brine, dried over MgSO₄ and concentrated under reduced pressure. The crude product was purified by flash chromatography on silica gel (Toluene/ethyl acetate, 8/2, v/v, UV) to afford 31 as a white solid (4.47 g, 88%). ¹H NMR (CDCl₃, 400 MHz): δ 8.11 (d, J=2.2 Hz, 1H), 7.55 (dd, J=8.2, 2.2 Hz, 1H), 7.12 (d, J=8.2 Hz, 1H), 2.92 (dd, J=16.3, 3.6 Hz, 1H), 2.71 (dd, J=13.0, 1.8 Hz, 1H), 2.60 (dd, J=16.3, 10.0 Hz, 1H), 2.34-2.25 (m, 2H), 1.13 (d, J=6.2 Hz, 3H). ¹³C NMR (CDCl₃, 101 MHz): δ 197.2, 142.5, 136.3, 133.8, 130.8, 129.9, 120.7, 46.9, 37.5, 30.4, 21.4. HRMS calculated for C₁₁H₁₂BrO [M+H]⁺: 239.006604. Found 239.006402. M.p.=59-61° C.

### 2-bromo-7-bromo-3-methyl-3,4-dihydronaphthalen-1(2H)-one (14)

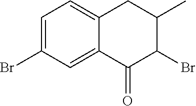

A solution of 31 (96 mg, 0.401 mmol, 1 equiv.) in dichloromethane (0.14 mL) was added dropwise to a solution of N-bromosuccinimide (71.5 mg, 0.401 mmol, 1 equiv.) and p-toluenesulfonic acid monohydrate (7.75 mg, 0.040 mmol, 0.1 equiv.) in dichloromethane (0.45 mL) at 0° C. The reaction mixture was stirred at reflux for 3.5 h. After addition of water, the organic layer was separated and the aqueous layer was extracted with dichloromethane. The combined organic layers were washed with saturated aqueous NaHCO₃ and brine, dried over MgSO₄ and concentrated under reduced pressure. The crude residue was purified by flash chromatography on silica gel (Toluene/cyclohexane, 8/2, v/v, UV) to afford 14 as yellowish oil and mixture of isomers (ratio A/B: 1.6/1, 103 mg, 81%). ¹H NMR (CDCl₃, 400 MHz): (*refers to the isomer B when unambiguous distinction is possible) δ 8.17 (d, J=2.2 Hz, 0.6H), 8.16* (d, J=2.2 Hz, 0.4H), 7.60 (td, J=8.2, 2.2 Hz, 1H), 7.14 (dd, J=8.1, 3.5 Hz, 1H), 4.50 (br dd, J=2.4, 0.8 Hz, 0.6H), 4.42* (d, J=8.0 Hz, 0.4H), 3.38* (dd, J=16.8, 4.0 Hz, 0.4H), 2.86 (m, 1H), 2.76-2.51 (m, 1.4H), 2.25 (dtdq, J=10.6, 6.4, 3.8, 2.3, 1.8 Hz, 0.6H), 1.21 (d, J=6.5 Hz, 1.8H), 1.18* (d, J=6.9 Hz, 1.2H). ¹³C NMR (CDCl₃, 101 MHz): (*refers to the isomer B when unambiguous distinction is possible) δ 189.8, 189.4*, 141.5, 139.9*, 137.2*, 136.9, 131.34, 131.28*, 131.1*, 130.98*, 130.95, 130.6, 121.1, 58.1, 58.6*, 36.9*, 34.4, 33.4*, 33.0, 19.0. HRMS calculated for C₁₁H₁₁Br₂O [M+H]⁺: 316.917116. Found 316.915206.

### 7-bromo-3-methylnaphthalen-1-ol (32)

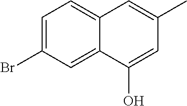

To a solution of 14 (90 mg, 0.28 mmol, 1 equiv.) in dimethylformamide (1.42 mL) was added lithium carbonate (23 mg, 0.31 mmol, 1.1 equiv.). The reaction mixture was stirred at 100° C. for 1.5 hours and then allowed to cool to room temperature. This reaction mixture was treated with ice-cold water and extracted with diethyl ether (3×). The combined extracts were washed a 1M solution of HCl, then with brine, dried over MgSO₄, and concentrated under reduced pressure. The crude was purified by flash chromatography on silica gel (Toluene/Cyclohexane, 8/2, v/v, UV) to afford 32 as a white solid (49 mg, 73%). ¹H NMR (CDCl₃, 400 MHz): δ 8.30 (s, 1H), 7.60-7.49 (m, 2H), 7.17 (s, 1H), 6.65 (s, 1H), 5.24 (s, 1H), 2.42 (s, 3H). ¹³C NMR (CDCl₃, 101 MHz): δ 150.4, 136.6, 133.4, 130.0, 128.8, 124.3, 123.9, 119.8, 118.4, 111.8, 21.9. HRMS calculated for CH₁₀BrO [M+H]⁺: 236.990953. Found 236.990412. M.p.=105-107° C.

### 6-bromo-2-methylnaphthalene-1,4-dione (2)

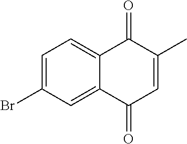

A solution of 32 (42 mg, 0.18 mmol, 1 equiv.) in a mixture of acetonitrile (2.5 mL) and water (0.9 mL) was prepared. At 0° C., (diacetoxyiodo)benzene (120 mg, 0.37 mmol, 2.1 equiv.) was added portionwise to the stirring solution. The mixture reaction was stirred for 30 minutes at 0° C. then at room temperature for 1 h. A saturated aqueous solution of NaHCO₃ was added to the yellow mixture and it was extracted with ethyl acetate. The organic phases were washed with brine, dried over MgSO₄ and concentrated under reduced pressure. The crude mixture was purified by flash chromatography on silica gel (Toluene/Cyclohexane, 8/2, v/v, UV) to give a yellow solid (39 mg, 88%). ¹H NMR (CDCl₃, 400 MHz): δ 8.16 (d, J=2.0 Hz, 1H), 7.94 (d, J=8.3 Hz, 1H), 7.83 (dd, J=8.3, 2.0 Hz, 1H), 6.83 (q, J=1.5 Hz, 1H), 2.19 (d, J=1.5 Hz, 3H). ¹³C NMR (CDCl₃, 101 MHz): δ 184.9, 183.8, 148.6, 136.7, 135.5, 133.4, 130.8, 129.34, 129.29, 128.4, 16.6. HRMS calculated for C₁₁H₈BrO₂ [M+H]⁺: 250.970218. Found 250.970402. M.p.=129-130° C.

### Example 6: Preparation of 6-chloromenadione

The 6-chloromenadione is prepared according to the following reaction scheme:

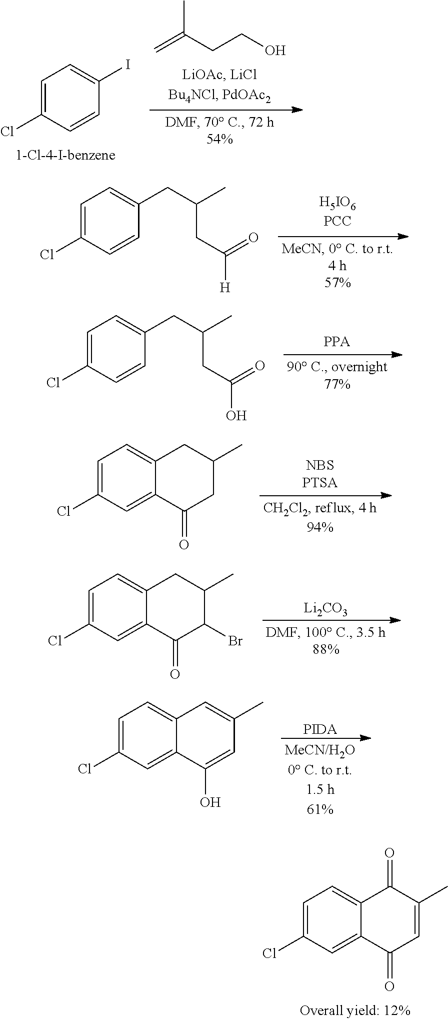

### 4-(4-chlorophenyl)-3-methyl-4-oxobut-2-enoic acid (15)

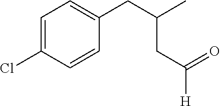

15 was synthetized according to Sleebs procedure. A mixture of 1-chloro-4-iodobenzene (10.0 g, 41.94 mmol, 1 equiv.), lithium acetate (6.89 g, 104.37 mmol, 2.49 equiv.), lithium chloride (1.78 g, 41.98 mmol, 1 equiv.), 3-methyl-3-buten-1-ol (4.25 mL, 41.94 mmol, 1 equiv.), tetrabutylammonium chloride hydrate (24.9 g, 83.97 mmol, 2 equiv.) and palladium diacetate (0.53 g, 2.37 mmol, 0.057 equiv.) in dimethylformamide (81 mL) was stirred at 70° C. for 72 h. After this time, the reaction mixture was quenched with a saturated aqueous solution of ammonium chloride. Then, the reaction mixture was extracted three times with diethyl ether. The combined organic layers were washed with brine, dried over MgSO₄ and concentrated under reduced pressure. The crude residue was purified by flash chromatography on silica gel (Cyclohexane, UV) to afford 15 as an orange oil. ¹H NMR (CDCl₃, 400 MHz): δ 9.75-9.71 (m, 1H), 7.30-7.25 (m, 2H), 7.13-7.07 (m, 2H), 2.60 (dd, J=13.5, 6.7 Hz, 1H), 2.51 (dd, J=13.5, 7.1 Hz, 1H), 2.46-2.38 (m, 1H), 2.38-2.31 (m, 1H), 2.27 (ddd, J=15.6, 7.2, 2.2 Hz, 1H), 0.98 (d, J=6.5 Hz, 3H). ¹³C NMR (CDCl₃, 101 MHz): δ 202.2, 138.6, 132.0, 130.6 (2C), 128.5 (2C), 50.2, 42.5, 30.1, 19.8.

### 4-(4-chlorophenyl)-3-methylbutanoic acid (16)

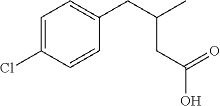

200 mL of acetonitrile was added to periodic acid (5.48 g, 24.05 mmol, 1.09 equiv.) and the resulting mixture was stirred for 15 min. At 0° C., a solution of 4-(4-bromophenyl)-3-methylbutanal (4.36 g, 22.17 mmol, 1 equiv.) in acetonitrile (15 mL) was added followed by pyridinium chlorochromate (0.095 g, 0.44 mmol, 0.02 equiv.) in 15 mL of acetonitrile. The reaction mixture was then stirred at room temperature for 4 h. After this time, ethyl acetate was added. The organic phase was washed with a 1:1 water/brine mixture, a saturated aqueous of sodium hydrogen sulfate solution and brine. It was then dried over MgSO₄ and concentrated under reduced pressure. The crude product was purified by flash chromatography on silica gel (Dichloromethane/methanol, gradient from 98/2 to 95/5, v/v, UV) to afford 16 as a brown oil. ¹H NMR (CDCl₃, 500 MHz): δ 7.43-7.39 (m, 2H), 7.27-7.23 (m, 2H), 2.79 (dd, J=13.5, 6.6 Hz, 1H), 2.64 (dd, J=13.5, 7.4 Hz, 1H), 2.50 (dd, J=14.7, 5.5 Hz, 1H), 2.41 (tt, J=12.6, 6.5 Hz, 1H), 2.34 (m, 1H), 1.12 (d, J=6.5 Hz, 3H). ¹³C NMR (CDCl₃, 126 MHz): δ 179.5, 138.6, 132.1, 130.7 (2C), 128.5 (2C), 42.3, 40.8, 32.1, 19.6. HRMS calculated for C₁₁H₁₂ClO₂ [M−H]⁻: 211.053131. Found 211.052909.

### 7-chloro-3-methyl-3,4-dihydronaphthalen-1(2H)-one (17)

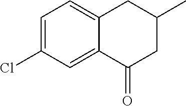

Polyphosphoric acid (25 mL) was poured in a 100 mL flask containing 4-(4-bromophenyl)-3-methylbutanoic acid (2.56 g, 12.04 mmol, 1 equiv.). The reaction mixture was stirred slowly overnight at 90° C. After TLC check, the mixture was poured on ice water until PPA was completely dissolved by using sonicator. Then, the aqueous phase was extracted with ethyl acetate (3 times). The reunited organic phases were washed with brine, dried over MgSO₄ and concentrated under reduced pressure. The crude product was purified by flash chromatography on silica gel (Toluene/ethyl acetate, gradient from 7/3 to 9/1, v/v, UV) to afford 17 as a brown solid (1.8 g, 77%). ¹H NMR (CDCl₃, 400 MHz): δ 7.95 (d, J=2.3 Hz, 1H), 7.40 (dd, AB syst, J=8.2, 2.3 Hz, 1H), 7.18 (d, AB syst, J=8.2 Hz, 1H), 2.94 (dd, J=16.2, 3.6 Hz, 1H), 2.72 (dd, J=13.0, 1.8 Hz, 1H), 2.63 (dd, J=16.4, 10.0 Hz, 1H), 2.36-2.24 (m, 2H), 1.13 (d, J=6.2 Hz, 3H). ¹³C NMR (CDCl₃, 101 MHz): δ 197.3, 142.1, 133.5, 133.4, 132.9, 130.5, 126.9, 46.9, 37.5, 30.4, 21.4. HRMS calculated for C₁₁H₁₂ClO [M+H]+: 195.0571. Found 195.0561. M.p.=49-51° C.

### 2-bromo-7-chloro-3-methyl-3,4-dihydronaphthalen-1(2H)-one (18)

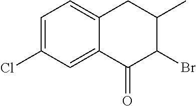

A solution of 17 (1.71 g, 8.78 mmol, 1 equiv.) in dichloromethane (3.1 mL) was added dropwise to a solution of N-bromosuccinimide (1.56 g, 8.78 mmol, 1 equiv.) and p-toluenesulfonic acid monohydrate (0.17 g, 0.88 mmol, 0.1 equiv.) in dichloromethane (9.8 mL) at 0° C. The reaction mixture was stirred at reflux for 4 h. After addition of water, the organic layer was separated and the aqueous layer was extracted with dichloromethane. The combined organic layers were washed with saturated aqueous NaHCO₃ and brine, dried over MgSO₄ and concentrated under reduced pressure. The product 18 was directly engaged in the next step without further purification (94% brut yield).

### 7-chloro-3-methylnaphthalen-1-ol (19)

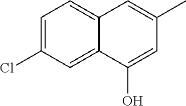

To a solution of 18 (2.25 g, 8.23 mmol, 1 equiv.) in dimethylformamide (41.1 mL) was added lithium carbonate (0.67 g, 9.05 mmol, 1.1 equiv.). The reaction mixture was stirred at 100° C. for 3 h and then allowed to cool to room temperature. This reaction mixture was treated with ice-cold water and extracted with diethyl ether (3×). The combined extracts were washed a 1 M solution of HCl, then with brine, dried over MgSO₄, and concentrated under reduced pressure. The crude was purified by flash chromatography on silica gel (Toluene/Cyclohexane, 8/2, v/v, UV) to afford 19 as an orange solid (1.4 g, 88%). ¹H NMR (CDCl₃, 400 MHz): δ 8.12 (s, 1H), 7.63 (d, J=8.7 Hz, 1H), 7.42-7.34 (m, 1H), 7.18 (s, 1H), 6.65 (s, 1H), 5.36 (s, 1H), 2.42 (s, 3H). ¹³C NMR (CDCl₃, 101 MHz): δ 150.4, 136.3, 133.1, 130.2, 128.6, 127.4, 123.4, 120.9, 119.6, 111.7, 21.7. HRMS calculated for C₁₁H₉ClO [M]: 192.0336. Found 192.0320. M.p.=105-106° C.

### 6-chloro-2-methylnaphthalene-1,4-dione (3)

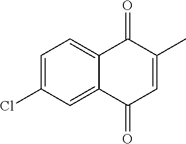

A solution of 19 (1.32 g, 6.85 mmol, 1 equiv.) in a mixture of acetonitrile (95.1 mL) and water (33.3 mL) was prepared. At 0° C., (diacetoxyiodo)benzene (4.63 g, 14.39 mmol, 2.1 equiv.) was added portionwise to the stirring solution. The mixture reaction was stirred for 30 minutes at 0° C. then at room temperature for 1 h. A saturated aqueous solution of NaHCO₃ was added to the yellow mixture and it was extracted with ethyl acetate. The organic phases were washed with brine, dried over MgSO₄ and concentrated under reduced pressure. The crude mixture was purified by flash chromatography on silica gel (Toluene/Cyclohexane, gradient from 7/3 to 9/1, v/v, UV) to give a yellow solid (860 mg, 61%). ¹H NMR (CDCl₃, 400 MHz): δ 8.04 (d, J=8.3 Hz, 1H), 8.01 (d, J=1.9 Hz, 1H), 7.67 (dd, J=8.3, 2.0 Hz, 1H), 6.85 (br d, J=1.0 Hz, 1H), 2.20 (d, J=0.8 Hz, 1H). ¹³C NMR (CDCl₃, 101 MHz): δ 184.6, 183.8, 148.6, 140.8, 135.6, 133.7, 133.5, 130.4, 128.4, 126.2, 16.6. HRMS calculated for C₁₁H₃ClO₂ [M+H]⁺: 207.0207. Found 207.0221. M.p.=115-116° C.

What is claimed is:

1. A process for the preparation of a compound having the following formula (I): 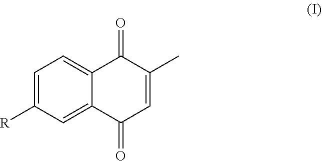
wherein R is selected from the group consisting of: halogen, (C₁-C₆)alkoxy, (C₁-C₆)alkyl, halo(C₁-C₆)alkyl, and halo(C₁-C₆)alkoxy,
said process comprising the following steps:
a step of intramolecular cyclisation of a compound having the following formula (II): 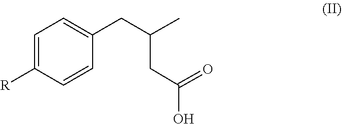
R being as defined above,
in order to obtain a compound having the following formula (III): 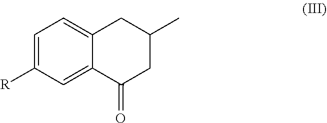
R being as defined above,
a step of bromination of the compound having the formula (III), in order to obtain a compound having the following formula (IV): 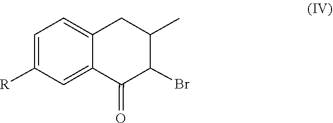
R being as defined above,
a step of aromatization of the compound having the formula (IV), in order to obtain a compound having the following formula (V): 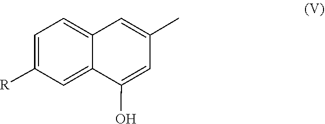
R being as defined above,
and
a step of oxidation of the compound having the formula (V), in order to obtain the compound of formula (I).

2. The process of claim 1, wherein R is selected from the group consisting of: F, Br, Cl, (C₁-C₆)alkoxy, (C₁-C₆)alkyl, and halo(C₁-C₆)alkyl.

3. The process of claim 1, wherein R is selected from the group consisting of: F, Br, Cl, OMe, Me, and CF₃.

4. The process of claim 1, wherein, when R is selected from the group consisting of: F, (C₁-C₆)alkoxy, (C₁-C₆)alkyl, halo(C₁-C₆)alkyl, and halo(C₁-C₆)alkoxy, the compound of formula (II) is prepared by a process comprising the following steps:
the reaction of glyoxylic acid with a compound having the following formula (VI): 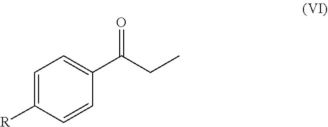
R being as defined above,
in order to obtain a compound having the following formula (VII): 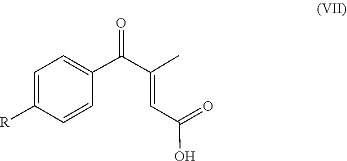
R being as defined above,
and
a step of hydrogenation of the compound having the formula (VII) in order to obtain the compound having the formula (II).

5. The process of claim 1, wherein, when R is Br or Cl, the compound of formula (II) is prepared by a process comprising the following steps:
the reaction of 3-methyl-3-buten-1-ol with a compound having the following formula (VIII): 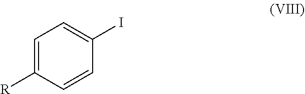
R being as defined above,
in order to obtain a compound having the following formula (IX): 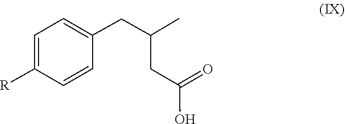
R being as defined above,
and
a step of oxidation of the compound having the formula (IX) in order to obtain the compound having the formula (II).

6. The process of claim 1, wherein the intramolecular cyclisation step is carried out in acidic conditions.

7. The process of claim 1, wherein the bromination step is carried out with the addition of a brominating agent.

8. The process of claim 1, wherein the aromatization step is carried out in the presence of Li₂CO₃.

9. The process of claim 1, wherein the oxidation step is carried out in the presence of an oxidant selected from the group consisting of: periodic acid (H₅IO₆), KMnO₄/H₂SO₄, and CrO₃/H₂SO₄.

10. The compound having the following formula (I): 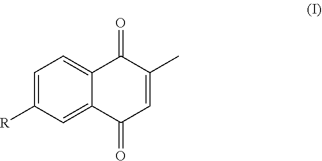
wherein R is selected from the group consisting of: Br, halo(C₁-C₆)alkyl, and halo(C₁-C₆)alkoxy.

11. The compound having the following formula (V): 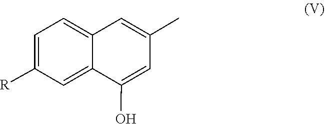
wherein R is selected from the group consisting of: F, Cl, (C₁-C₆)alkoxy, halo(C₁-C₆)alkyl, and halo(C₁-C₆)alkoxy.

12. The compound having the following formula (IV): 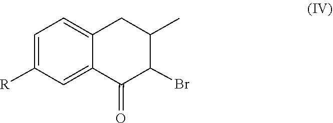
wherein R is selected from the group consisting of: F, Cl, Br, (C₁-C₆)alkoxy, (C₁-C₆)alkyl, halo(C₁-C₆)alkyl, and halo(C₁-C₆)alkoxy.

13. The compound having the following formula (III): 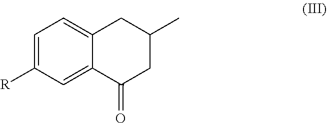
wherein R is selected from the group consisting of: F, Cl, halo(C₁-C₆)alkyl, and halo(C₁-C₆)alkoxy.

14. The compound having the following formula (II): 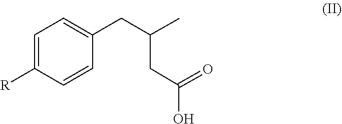
wherein R is selected from the group consisting of: Cl, (C₁-C₆)alkoxy, halo(C₁-C₆)alkyl, and halo(C₁-C₆)alkoxy.

15. The compound having the following formula (VII): 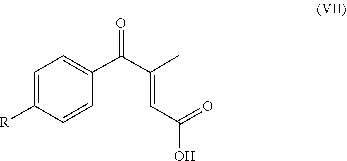
wherein R is selected from the group consisting of: halo(C₁-C₆)alkyl, and halo(C₁-C₆)alkoxy.

16. A compound selected from the following compounds:

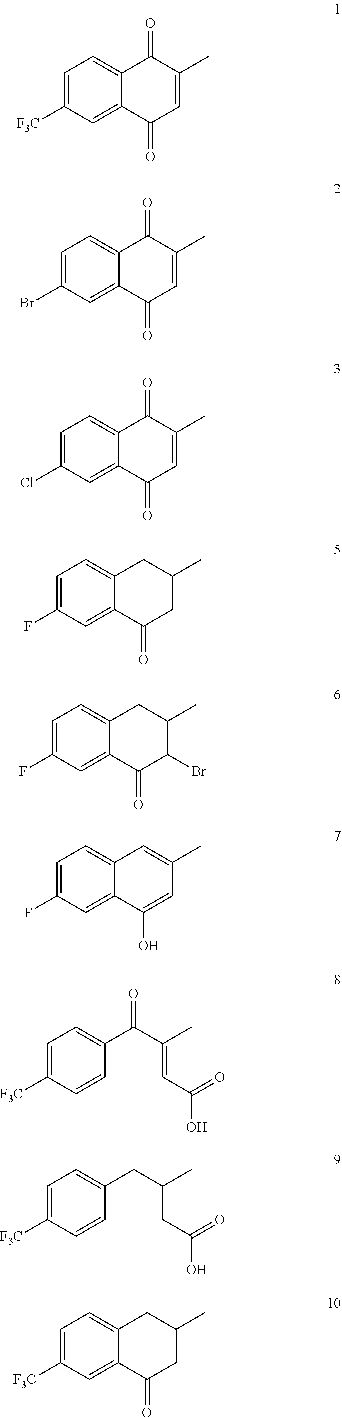

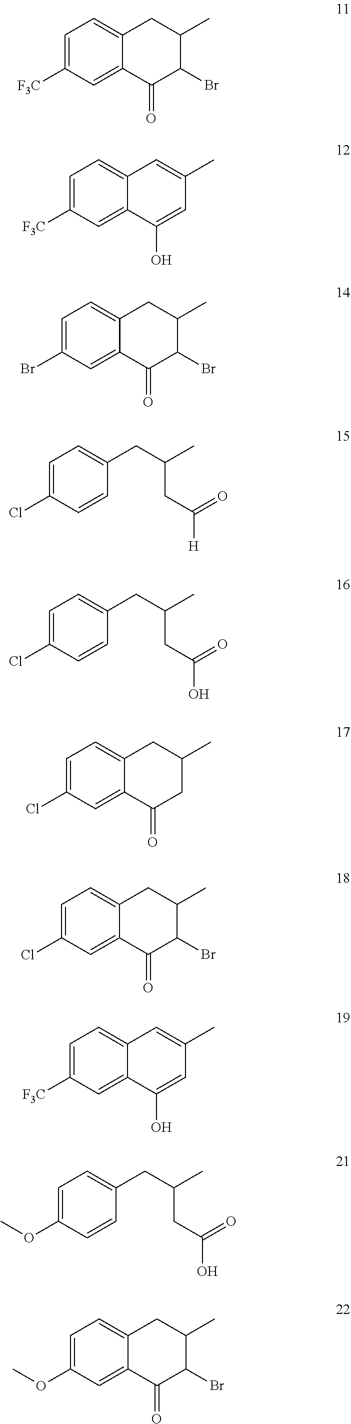

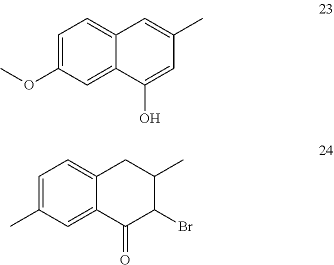

17. The process according to claim 6, wherein the intramolecular cyclisation step is carried out in acidic conditions in the presence of polyphosphoric acid or trifluoroacetic anhydride/triflic acid.

18. The process according to claim 7, wherein the brominating agent is N-bromosuccinimide.

19. The compound according to claim 10, wherein R is Br or CF₃.

20. The compound according to claim 11, wherein R is selected from the group consisting of: F, CF₃, Cl and OMe.

21. The compound according to claim 12, wherein R is selected from the group consisting of: F, Br, Cl, CF₃, Me and OMe.

22. The compound according to claim 13, wherein R is selected from the group consisting of: F, Cl and CF₃.

23. The compound according to claim 14, wherein R is selected from the group consisting of: Cl, CF₃ and OMe.

24. The compound according to claim 15, wherein R is CF₃.
